(19) United States
(12) Patent Application Publication
(10) Pub. No.: US 2026/0007678 A1
(43) Pub. Date: Jan. 8, 2026

(54) COMBINATION OF ALLOSTERIC AND ORTHOSTERIC EGFR INHIBITORS FOR THE TREATMENT OF CANCER

(71) Applicant: Hoffmann-La Roche Inc., Little Falls, NJ (US)

(72) Inventors: Georg JAESCHKE, Basel (CH); Bruno MARTOGLIO, Eschenbach SG (CH); Yvonne Alice NAGEL, Basel (CH); Christa Ulrike OBST-SANDER, Reinach BL (CH); Antonio RICCI, Biel-Benken (CH)

(73) Assignee: Hoffmann-La Roche Inc., Little Falls, NJ (US)

(21) Appl. No.: 18/865,274

(22) PCT Filed: May 11, 2023
(86) PCT No.: PCT/EP2023/062530

§ 371 Date: Nov. 12, 2024

(30) Foreign Application Priority Data
May 13, 2022 (EP) 22173211.8
May 25, 2022 (EP) 22175302.3

(51) Int. Cl. A61K 31/5377; A61K 31/454; A61K 31/506; A61K 31/517; A61P 35/00

(52) U.S. Cl. CPC A61K 31/5377; A61K 31/454; A61K 31/506; A61K 31/517; A61P 35/00

(57) ABSTRACT

The present invention is directed to the combination therapy of cancer with an allosteric EGFR inhibitor and an orthosteric EGFR inhibitor, as well as uses and pharmaceutical compositions thereof.

The present invention relates to a novel combination of an orthosteric EGFR inhibitor with a selective allosteric EGFR inhibitor of T790M/L858R, T790M/L858R/C797S, L858R, L858R/C797S containing EGFR mutants, as well as uses and pharmaceutical compositions thereof.

The present invention provides in particular a combination of an allosteric EGFR inhibitor and an orthosteric EGFR inhibitor, wherein the allosteric EGFR inhibitor is a compound of formula (I)

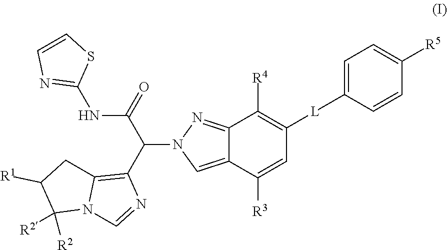

wherein
L is a bond or alkynylene;
R¹ is hydrogen or halogen;
R² and R^{2′} are independently selected from hydrogen and alkyl;
or R² and R^{2′}, together with the carbon atom to which they are attached, form cycloalkyl;
R³ is hydrogen, halogen or haloalkyl;
R⁴ is alkyl or halogen;
R⁵ is (heterocycloalkyl)alkylene or heterocycloalkyl, wherein (heterocycloalkyl)alkylene is optionally substituted with one or two substituents independently selected from R⁶, and wherein heterocycloalkyl is optionally substituted with one or two substituents independently selected from R⁷;
R⁶ is at each instance independently selected from alkyl, cycloalkyl, hydroxy and hydroxyalkyl; and
R⁷ is at each instance independently selected from alkyl, cycloalkyl, hydroxy and hydroxyalkyl;
or a pharmaceutically acceptable salt thereof.

The HER family receptor tyrosine kinases are mediators of cell growth, differentiation and survival. The receptor family includes four distinct members, i.e. epidermal growth factor receptor (EGFR, ErbB1, or HER1) HER2 (ErbB2), HER3 (ErbB3) and HER4 (ErbB4). Upon ligand binding the receptors form homo and heterodimers and subsequent activation of the intrinsic tyrosine kinase activity leads to receptor auto-phosphorylation and the activation of downstream signaling molecules (Yarden, Y., Sliwkowski, MX. Untangling the ErbB signalling network. Nature Review Mol Cell Biol. 2001 February; 2(2): 127-37). De-regulation of EGFR by overexpression or mutation has been implicated in many types of human cancer including colorectal, pancreatic, gliomas, head and neck and lung cancer, in particular non-small cell lung cancer (NSCLC) and several EGFR targeting agents have been developed over the years (Ciardiello, F., and Tortora, G. (2008). EGFR antagonists in cancer treatment. The New England journal of medicine 358, 1160-1174). Erlotinib (Tarceva®), a reversible inhibitor of the EGFR tyrosine kinase was approved in numerous countries for the treatment of recurrent NSCLC.

An impressive single agent activity of EGFR tyrosine kinase inhibitors is observed in a subset of NSCLC patients whose tumors harbor somatic kinase domain mutations, whereas clinical benefit in wild-type EGFR patients is greatly diminished (Paez, J. et al. (2004). EGFR mutations in lung cancer: correlation with clinical response to gefitinib therapy. Science (New York, NY 304, 1497-1500)). The most common somatic mutations of EGFR are exon 19 deletions with delta 746-750 the most prevalent mutation and the exon 21 amino acid substitutions with L858R the most frequent mutation (Sharma S V, Bell D W, Settleman J, Haber D A. Epidermal growth factor receptor mutations in lung cancer. Nat Rev Cancer. 2007 March; 7(3): 169-81).

Treatment resistance arises frequently, often due to the secondary T790M mutation within the ATP site of the receptor. Some of the developed mutant-selective irreversible inhibitors, such as for instance osimertinib, almonertinib, lazertinib and furmonertinib, are highly active against the T790M mutant, but their efficacy can be compromised by acquired mutation of C797S, that is the cysteine residue with which they form a key covalent bond (Thress, K. S. et al. Acquired EGFR C797S mutation mediates resistance to AZD9291 in non-small cell lung cancer harboring EGFR T790M. Nat. Med. 21, 560-562 (2015)). C797S mutation was further reported by Wang to be a major mechanism for resistance to T790M-targeting EGFR inhibitors (Wang et al. EGFR C797S mutation mediates resistance to third-generation inhibitors in T790M-positive non-small cell lung cancer, J Hematol Oncol. 2016; 9:59). Additional mutations that cause resistance to Osimertinib are described by Yang, for example L718Q. (Yang et al, Investigating Novel Resistance Mechanisms to Third-Generation EGFR Tyrosine Kinase Inhibitor Osimertinib in Non-Small Cell Lung Cancer Patients, Clinical Cancer Research, DOI: 10.1158/1078-0432.CCR-17-2310) Lu et al. (Targeting EGFRL858R/T790M and EGFRL858R/T790M/C797S resistance mutations in NSCLC: Current developments in medicinal chemistry, Med Res Rev 2018; 1-32) report in a review article on Targeting EGFRL858R/T790M and EGFRL858R/T790M/C797S resistance mutations in NSCLC treatment.

As most available EGFR tyrosine kinase inhibitors target the ATP-site of the kinase, there is a need for new therapeutic agents that work differently, for example through targeting drug-resistant EGFR mutants.

Recent studies suggest that purposefully targeting allosteric sites might lead to mutant-selective inhibitors (Jia et al. Overcoming EGFR (T790M) and EGFR (C797S) resistance with mutant-selective allosteric inhibitors, June 2016, Nature 534, 129-132)

Accordingly, there is a need for the generation of selective molecules that specifically inhibit T790M/L858R, T790M/L858R/C797S, L858R, L858R/C797S containing EGFR mutants. Such selective inhibitors could be useful for the therapeutic and/or prophylactic treatment of cancer, in particular T790M and C797S containing EGFR mutants.

Importantly, such selective allosteric inhibitors were designed to treat cancer characterized by EGFR mutants that became resistant to orthosteric EGFR inhibitors. The rational being that once drug-resistance occurred the treatment could be switched from an orthosteric EGFR inhibitor to an allosteric EGFR inhibitor that was specifically designed for targeting mutants bearing T790M and/or C797S mutations.

The present invention relates to a combination of an orthosteric EGFR inhibitor with a selective allosteric EGFR inhibitor that corresponds to the compound of formula (I). The compound of formula (I) efficiently targets T790M/L858R, T790M/L858R/C797S, L858R, L858R/C797S mutations and has low hepatic clearance in vitro (Tables 1 and 2). Importantly, the compound of formula (I) also efficiently targets the L858R/C797S mutation in vivo in a Ba/F3 based allograft (FIG. 1), a mutation that causes resistance to orthosteric EGFR inhibitors. Surprisingly, the combined treatment with a clinically relevant dose of the approved orthosteric EGFR inhibitor osimertinib and an allosteric EGFR inhibitor of formula (I) drastically improved tumour regression in mice with NCI-H1975 xenograft, which is an established model of EGFR-driven NSCLC harboring the EGFR^{L858R/T790M} double mutation (FIG. 2). Importantly, the binding of an EGFR inhibitor of formula (I) increases the residence time of the orthosteric EGFR inhibitor osimertinib around 120 fold, and thus accelerates the rate with which osimertinib covalently bonds to EGFR (FIG. 3). This surprising positive cooperativity between an allosteric EGFR inhibitor of formula (I) and an orthosteric EGFR inhibitor has not been previously reported and offers promising new therapeutic strategies for patients with EGFR-related cancer, in particular non-small cell lung cancer.

### BRIEF DESCRIPTION OF FIGURES

FIG. 1: (A) Anti-tumor activity of compound Example 6 (square) and compound Example 3 (triangle) in an Ba/F3 EGFR^{L858R/C797S} allograft at 10 mpk and 30 mpk, respectively, BID po. Black arrows indicate dosings. A tumor control ratio (TCR) below 1.0 indicated tumor growth inhibition, with confidence interval (CI) reflecting the data distribution. An upper CI below 1.0 confirms statistical significance. No significant changes in body weight occurred for the different cohorts.

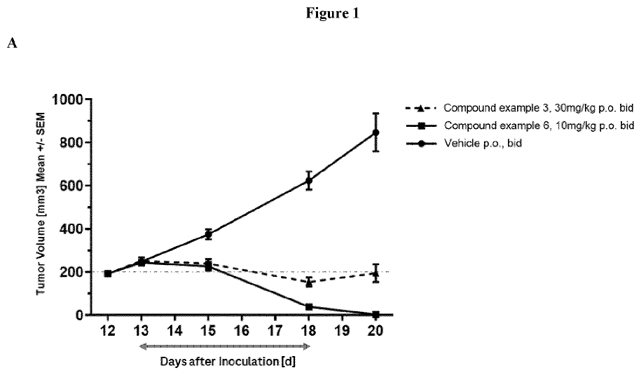

FIG. 2 (A) Anti-tumor activity of compound Example 6 alone and in combination with osimertinib in NCI-H1975 NSCLC-derived tumors (EGFR^{L858R/T790M} xenograft) in mice. Black arrow indicates dosings. A TCR below 1.0 indicated tumor growth inhibition, with CI reflecting the data distribution. An upper CI below 1.0 confirms statistical significance. No significant changes in body weight occurred for the different cohorts. (B) Anti-tumor activity of compound Example 1 alone and in combination with osimertinib in NCI-H1975 NSCLC-derived tumors (EGFR^{L858R/T790M} xenograft) in mice. Black arrow indicates dosings. A TCR below 1.0 indicated tumor growth inhibition, with CI reflecting the data distribution. An upper CI below 1.0 confirms statistical significance.

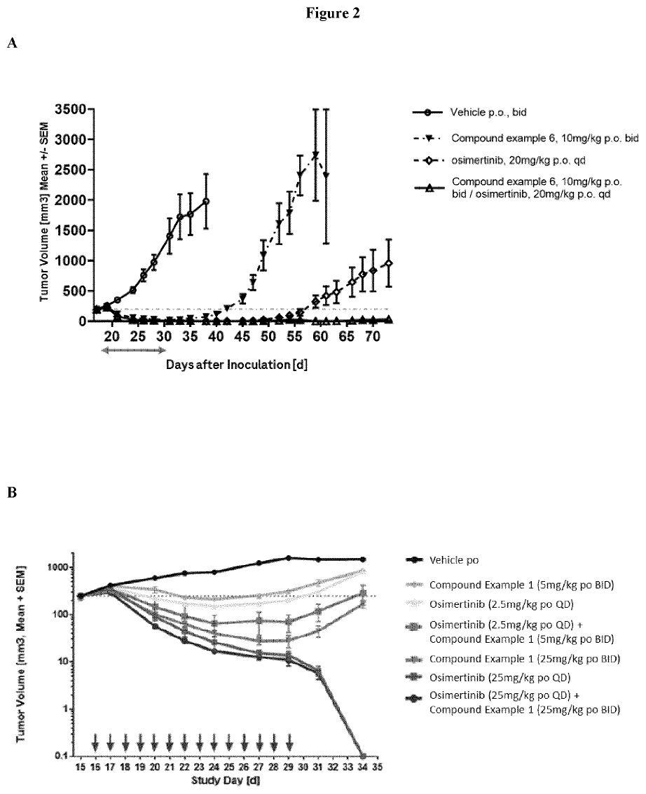

FIG. 3 (A) Binding kinetics measured by stopped-flow fluorescence. Osimertinib quenches the TRp-fluorescence of EGFR when bound. (B) Binding kinetics directly show that osimertinib reacts faster with the EGFR kinase domain (L858R) in presence of the allosteric inhibitor compound Example 1, indicating positive cooperativity. (C) Global fitting of the kinetic data reveals the mechanism of positive cooperativity: compound Example 1 increases the residence time of osimertinib around 120 fold, and thus accelerates the rate with which the vital covalent bond is formed.

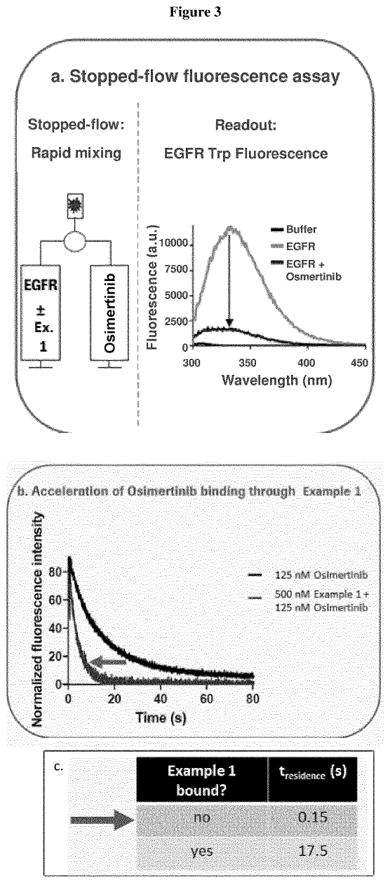

The term “inhibitor” denotes a compound which competes with, reduces or prevents the binding of a particular ligand to particular receptor, or which reduces or prevents the function of a particular protein. In particular, an inhibitor as used therein refers to compounds which target, decrease or inhibit EGFR activity, particular inhibitors have an IC50 value below 1 μM, below 500 nM, below 200 nM, below 100 nM, below 50 nM, below 25 nM, below 10 nM, below 5 nM, 2 nM or below 1 nM. In some embodiments of the invention the term “EGFR inhibitor” refers to compounds that decrease EGFR kinase activity at least about 10%, at least about 20%, at least about 30%, at least about 40%, at least about 50%, at least about 60%, at least about 70%, at least about 75%, at least about 80%, at least about 85%, at least about 90%, at least about 95% or at least about 99%. The term “orthosteric EGFR inhibitor” relates to an EGFR inhibitor that binds close to the active site, such as for instance erlotinib, geftinib, osimertinib, almonertinib, lazertinib and furmonertinib, or a pharmaceutically acceptable salt thereof, in particular a mesylate salt. Non-limiting examples of orthosteric EGFR inhibitors include cetuximab (Erbitux®), panitumumab (Vectibix®), osimertinib (merelectinib, Tagrisso®), erlotinib (Tarceva®), gefitinib (lressa®), necitumumab (Portrazza™), neratinib (Nerlynx®), lapatinib (Tykerb®), vandetanib (Caprelsa®) and brigatinib (Alunbrig®).

The term “IC50” refers to the concentration of a particular compound required to inhibit 50% of a specific measured activity.

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert.-butyl, sec.-butyl, the isomeric pentyls, the isomeric hexyls, the isomeric heptyls and the isomeric octyls, particularly methyl, ethyl, propyl, butyl and pentyl. Particular examples of “alkyl” are methyl, ethyl, propyl, isopropyl, and tert.-butyl. Methyl is a particular example of “alkyl” in the compound of formula (I).

The term “alkoxy” or “alkyloxy”, alone or in combination, signifies a group of the formula alkyl-O— in which the term “alkyl” has the previously given significance, such as methoxy, ethoxy, n-propoxy, isopropoxy, n-butoxy, isobutoxy, sec-butoxy and tert-butoxy. Particular examples of “alkoxy” are methoxy, ethoxy and tert-butoxy.

The term “alkylene”, alone or in combination, denotes a linear saturated divalent hydrocarbon group of 1 to 7 carbon atoms or a divalent branched saturated divalent hydrocarbon group of 3 to 7 carbon atoms. Examples of alkylene groups include methylene, ethylene, propylene, 2-methylpropylene, butylene, 2-ethylbutylene, pentylene, hexylene. A particular example of “alkylene” is methylene.

The term “alkynylene”, alone or in combination, denotes a linear divalent hydrocarbon chain of 2-6 carbon atoms or a branched divalent hydrocarbon chain of 3-6 carbon atoms with at least one triple bond. Exemplary alkynylene include ethynylene, 2,2-dimethylethynylene, propynylene, 2-methylpropynylene, butynylene, and pentynylene. A particular example of “alkynylene” is ethynylene.

The term “oxy”, alone or in combination, signifies the —O— group.

The terms “halogen” or “halo”, alone or in combination, signifies fluorine, chlorine, bromine or iodine and particularly fluorine or chlorine. A particular “halogen” or “halo” is fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. Particular examples of “haloalkyl” are difluoromethyl and trifluoromethyl.

The terms “hydroxyl” and “hydroxy”, alone or in combination, signify the —OH group.

The term “carbonyl”, alone or in combination, signifies the —C(O)— group.

The term “heterocycloalkyl”, alone or in combination, denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Bicyclic means consisting of two cycles having one or two ring atoms in common. Examples of “heterocycloalkyl” are morpholinyl, piperidinyl, azetidinyl and piperazinyl, Particular examples of “heterocycloalkyl” are piperidinyl and morpholinyl.

The term “cycloalkyl”, alone or in combination, denotes a monovalent saturated cyclic hydrocarbon group of 3 to 8 ring carbon atoms. Examples of “cycloalkyl” are cyclopropyl, cyclobutanyl, cyclopentyl, cyclohexyl or cycloheptyl. A particular example of the “cycloalkyl” group is cyclopropyl.

The terms “piperidinyl” and “piperidyl” are interchangeable and signify, alone or in combination, a saturated monocycle comprising 5 carbon ring atoms and one nitrogen ring atom.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compound of formula (I) are the hydrochloride salts, methanesulfonic acid salts and citric acid salts. Particular pharmaceutically acceptable salts of osimertinib are methanesulfonic acid salts, also referred to as mesylate salts.

In one embodiment the invention provides a kit comprising an orthosteric EGFR inhibitor and an allosteric EGFR inhibitors of formula (I) as described therein, prescribing information also known as “leaflet”, a blister package or bottle (HDPE or glass) and a container. The prescribing information preferably includes the advice to a patient regarding the administration of the combination of the orthosteric EGFR inhibitor and allosteric EGFR inhibitor treatment as described herein;

According to the Cahn-Ingold-Prelog Convention the asymmetric carbon atom can be of the “R” or “S” configuration. The compound of formula (I) can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

Furthermore, the invention includes all substituents in its corresponding deuterated form, wherever applicable, of the compound of formula (I).

In the embodiments, where optically pure enantiomers are provided, optically pure enantiomer means that the compound contains >90% of the desired isomer by weight, particularly >95% of the desired isomer by weight, or more particularly >99% of the desired isomer by weight, said weight percent based upon the total weight of the isomer(s) of the compound. Chirally pure or chirally enriched compound may be prepared by chirally selective synthesis or by separation of enantiomers. The separation of enantiomers may be carried out on the final product or alternatively on a suitable intermediate.

The compound of formula (I) and its pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays), rectally (e.g. in the form of suppositories) or topical ocularly (e.g. in the form of solutions, ointments, gels or water soluble polymeric inserts). However, the administration can also be effected parenterally, such as intramuscularly, intravenously, or intraocularly (e.g. in the form of sterile injection solutions).

The compound of formula (I) and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The following examples illustrate the present invention without limiting it, but serve merely as representative thereof. The pharmaceutical compositions conveniently contain about 1-500 mg, particularly 1-100 mg, of a compound of formula (I). The pharmaceutical compositions conveniently contain about 1-500 mg, particularly 1-100 mg, of a compound of formula (II). In certain embodiments the pharmaceutical compositions containing a compound of formula (I) contains in addition about 1-500 mg, particularly 80 mg, of an orthosteric EGFR inhibitor in a fixed-dose combination.

Non-limiting examples of compositions according to the invention are:

### Preparation of Pharmaceutical Compositions Comprising the Compound of the Invention

Tablets of the following composition are manufactured in the usual manner:

mg/tablet

| Ingredient | 5 | 25 | 100 | 500 |
|---|---|---|---|---|
| Compound of formula (I) | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

Capsules of the following composition are manufactured in the usual manner:

mg/capsule

| Ingredient | 5 | 25 | 100 | 500 |
|---|---|---|---|---|
| Compound of formula (I) | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

A compound of formula (I) lactose and corn starch are firstly mixed in a mixer and then in a comminuting machine. The mixture is returned to the mixer; the talc is added thereto and mixed thoapproximatively. The mixture is filled by machine into suitable capsules, e.g. hard gelatin capsules.

Injection solutions of the following composition are manufactured in the usual manner:

| Ingredient | mg/injection solution. |
|---|---|
| Compound of formula (I) | 3 |
| Polyethylene Glycol 400 | 150 |
| acetic acid | q.s. ad pH 5.0 |
| water for injection solutions | ad 1.0 ml |

### EXAMPLES

### Abbreviations

AcOH=acetic acid; ATP=adenosine triphosphate; BID=twice daily; CAS=chemical abstract service; CDI=1,1′-carbonyldiimidazole; Clᵢₙₜ=intrinsic clearance; Cₘₐₓ=maximum plasma concentration; D=study day; DCM=dichloromethane; DME=dimethoxyethane; DMF=dimethylformamide; DMSO=diemethyl sulfoxide; ESI=electrospray ionization; EtOAc=ethyl acetate; EtOH=ethanol; HATU=hexafluorophosphate azabenzotriazole tetramethyl uronium; LDA=lithium diisopropylamide; MeOH=methanol; MS=mass spectrometry; NMR=nuclear magnetic resonance; PO) orally, per os; QD=once daily; RT=room temperature; SC=subcutaneously; THF=tetrahydrofuran.

The following examples are provided for illustration of the invention. They should not be considered as limiting the scope of the invention, but merely as being representative thereof.

In case the preparative examples are obtained as a mixture of enantiomers, the pure enantiomers can be obtained by methods described herein or by methods known to those skilled in the art, such as e.g. chiral chromatography or crystallization.

### Example 1

### 2-[4,7-Dichloro-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide

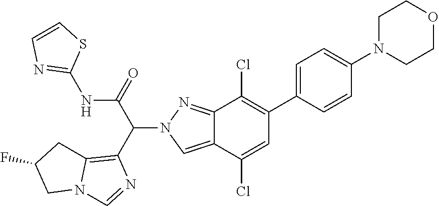 [SMILES: O=C(Nc1nccs1)C(c1ncn2c1C[C@@H](F)C2)n1cc2c(Cl)cc(-c3ccc(N4CCOCC4)cc3)c(Cl)c2n1]

### Step 1:4-Bromo-3,6-dichloro-2-fluorobenzaldehyde

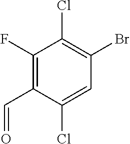 [SMILES: O=Cc1c(Cl)cc(Br)c(Cl)c1F]

A solution of 1-bromo-2,5-dichloro-3-fluorobenzene (9.41 g, 38.6 mmol) in tetrahydrofuran (70 ml) was cooled in a dry ice/acetone bath. LDA, 2 mol/l in THF (21.2 ml, 42.5 mmol, 1.1 equiv.) was added and the mixture was stirred at −75° C. for 20 minutes. N,N-Dimethylformamide (2.82 g, 3.0 ml, 38.6 mmol, 1 equiv.) was added dropwise and stirred for 1 hour. A solution of acetic acid in ether (1:1, 10 ml) was added. The mixture was allowed to warm to room temperature. Water was added and the mixture was extracted with ethyl acetate. The organic layers were washed with water, dried (MgSO₄), filtered and concentrated in vacuo to give the crude title compound (quantitative yield) as light yellow solid. The compound was used for the next step without further purification.

### Step 2:6-Bromo-4,7-dichloro-1H-indazole

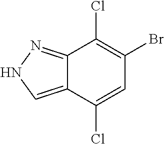 [SMILES: Clc1cc(Br)c(Cl)c2n[nH]cc12]

To a solution of 4-bromo-3,6-dichloro-2-fluorobenzaldehyde (Example 1, step 1) (10.5 g, 38.6 mmol) in dioxane (50 ml) was added hydrazine hydrate (3.86 g. 3.78 ml, 77.2 mmol, 2.0 equiv.). The mixture was stirred at room temperature for 3 days. Hydrazine hydrate (386 mg, 0.38 ml, 7.72 mmol, 0.2 equiv.) was added and the mixture was warmed to 70° C. for 7 hours. After cooling to room temperature water was added and the precipitated solid was collected by filtration. To the solid was added a small amount of acetonitrile and stirred for 2 hours. The solid was collected by filtration, washed with a small amount of acetonitrile and dried to give the title compound (7.8 g, 76% yield) as off-white solid. m/z 267.0/269.0, [M+H]⁺, ESI pos, Br isotopes.

### Step 3: Ethyl 2-(6-bromo-4,7-dichloro-indazol-2-yl)acetate

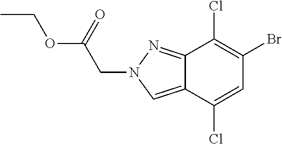 [SMILES: CCOC(=O)Cn1cc2c(Cl)cc(Br)c(Cl)c2n1]

To a solution of 6-bromo-4,7-dichloro-1H-indazole (Example 1, step 2) (7.84 g, 29.5 mmol, Eq: 1) in N,N-dimethylacetamide (11.5 mL) was added ethyl 2-bromoacetate (9.85 g, 6.53 ml, 59 mmol, 2.0 equiv.). The reaction mixture was stirred for 16 hours at 100° C. Ice was added and the precipitated solid was collected by filtration and washed with water. The compound was crystallized from boiling ethanol. The solid was collected by filtration, washed with a small amount of ethanol and dried to give the title compound as a white solid (7.5 g, 70% yield). m/z 353.0, 355.0, [M+H]⁺, ESI pos, Br isotopes.

### Step 4: tert-Butyl (2S,4R)-2-[2-(6-bromo-4,7-dichloro-indazol-2-yl)-3-ethoxy-3-oxo-propanoyl]-4-fluoro-pyrrolidine-1-carboxylate

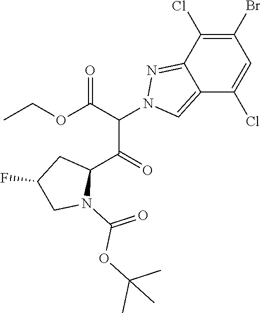 [SMILES: CCOC(=O)C(C(=O)[C@@H]1C[C@@H](F)CN1C(=O)OC(C)(C)C)n1cc2c(Cl)cc(Br)c(Cl)c2n1]

A solution of (2S,4R)-1-(tert-butoxycarbonyl)-4-fluoropyrrolidine-2-carboxylic acid (2.34 g, 10 mmol, 1.55 equiv.) in tetrahydrofuran (11 ml) was cooled in an ice bath. Carbonyldiimidazole (1.63 g, 10 mmol, 1.55 equiv.) was added. The cooling bath was removed and the mixture was stirred for 3 hours to give solution A. A solution of ethyl 2-(6-bromo-4,7-dichloro-indazol-2-yl)acetate (Example 1, step 3) (2.28 g, 6.5 mmol) in tetrahydrofuran (11 ml) was cooled to −70° C. LDA, 2 mol/l in tetrahydrofuran (5.0 ml, 10 mmol, 1.55 equiv.) was added dropwise within 5 min. The mixture was stirred for 30 minutes at −70° C. Solution A was added dropwise within 5 minutes. The mixture was allowed to warm to room temperature in the cooling bath overnight. After addition of saturated aqueous NH₄Cl-solution, the mixture was extracted twice with ethyl acetate. The organic layers were washed with water, combined, dried over sodium sulphate and concentrated to dryness to give the crude title compound (quantitative yield) which was used for the next step without further purification. m/z 566.1/568.1, [M+H]⁺, ESI pos, Br isotopes.

### Step 5: Ethyl 2-(6-bromo-4,7-dichloro-indazol-2-yl)-2-[(6R)-6-fluoro-3-thioxo-2,5,6,7-tetrahydropyrrolo[1,2-c]imidazol-1-yl]acetate

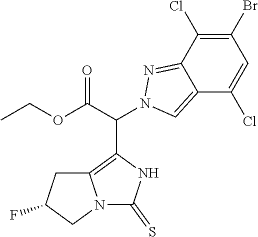 [SMILES: CCOC(=O)C(c1[nH]c(=S)n2c1C[C@@H](F)C2)n1cc2c(Cl)cc(Br)c(Cl)c2n1]

A solution of tert-butyl (2S,4R)-2-[2-(6-bromo-4,7-dichloro-indazol-2-yl)-3-ethoxy-3-oxo-propanoyl]-4-fluoro-pyrrolidine-1-carboxylate (Example 1, step 4) (4.23 g, 6.41 mmol) in HCl, 4M in dioxane (11 ml) was stirred for 1 hour at room temperature. The mixture was concentrated to dryness. The residue was dissolved in ethanol (37 ml), potassium thiocyanate (829 mg, 8.53 mmol, 1.33 equiv.) and HCl, 1 M in ethanol (12.8 ml) were added and stirred 40 hours at room temperature. Water was added and the mixture was extracted with ethyl acetate. The organic layers were washed with water, dried over MgSO₄, filtered, concentrated and dried to give the crude title compound (2.5 g, 76% yield) which was used for the next step without further purification. m/z 509.0/511.0, [M+H]⁺, ESI pos, Br isotopes.

### Step 6: Ethyl 2-(6-bromo-4,7-dichloro-indazol-2-yl)-2-[(6R)-6-fluoro-6,7-dihydro-5H-pyrrolo[1,2-c]imidazol-1-yl]acetate

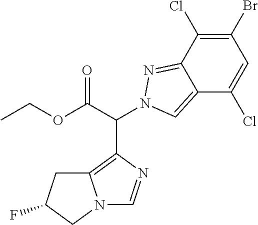 [SMILES: CCOC(=O)C(c1ncn2c1C[C@@H](F)C2)n1cc2c(Cl)cc(Br)c(Cl)c2n1]

A solution of ethyl 2-(6-bromo-4,7-dichloro-indazol-2-yl)-2-[(6R)-6-fluoro-3-thioxo-2,5,6,7-tetrahydropyrrolo[1,2-c]imidazol-1-yl]acetate (Example 1, step 5) (1.46 g, 2.88 mmol) in acetic acid (10 ml) was cooled to 10° C. Hydrogen peroxide, 35% (1.12 g, 1.01 ml, 11.5 mmol, 4 equiv.) was added dropwise. The reaction mixture was stirred for 1 hour at room temperature. The excess of hydrogen peroxide was destroyed by addition of saturated sodium sulfit solution. After addition of some water (just enough to dissolve all salts) and ethyl acetate the mixture was brought to pH 9 by careful addition of solid sodium carbonate. The mixture was extracted with ethyl acetate. The organic layers were washed with water, dried over sodium sulphate and concentrated. The product was purified by chromatography (SiO₂, 0-100% ethyl acetate in heptane) to give the title compound (0.81 g, 58% yield) as light brown solid. m/z 475.0/477.0, [M+H]⁺, ESI pos, Br isotopes.

### Step 7: Ethyl 2-[4,7-dichloro-6-(4-morpholinophenyl)indazol-2-yl]-2-[(6R)-6-fluoro-6,7-dihydro-5H-pyrrolo[1,2-c]imidazol-1-yl]acetate

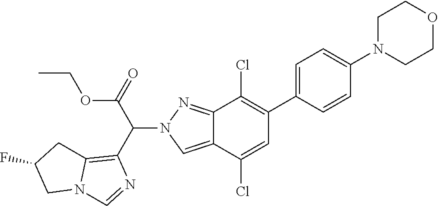 [SMILES: CCOC(=O)C(c1ncn2c1C[C@@H](F)C2)n1cc2c(Cl)cc(-c3ccc(N4CCOCC4)cc3)c(Cl)c2n1]

Ethyl 2-(6-bromo-4,7-dichloro-indazol-2-yl)-2-[(6R)-6-fluoro-6,7-dihydro-5H-pyrrolo[1,2-c]imidazol-1-yl]acetate (Example 1, step 6) (100 mg, 0.21 mmol), (4-morpholinophenyl) boronic acid (130 mg, 0.63 mmol, 3 equiv.) and cesium carbonate (205 mg, 0.63 mmol, 3 equiv.) were mixed with toluene (3.0 ml), degassed by bubbling argon through the mixture under ultra sonic treatment. [1,1′-Bis(diphenylphosphino) ferrocene]dichloropalladium (II) (15 mg, 0.02 mmol, 0.1 equiv.) was added and the mixture was stirred for 30 minutes at 110° C. in a sealed tube. The mixture was cooled to room temperature, diluted with ethyl acetate, washed with half concentrated sodium carbonate solution, dried over sodium sulphate and concentrated. The crude material was purified by flash chromatography (SiO₂, 0% to 40% methanol in ethyl acetate) to give the title compound (82 mg, 69% yield) as light brown amorphous solid. m/z 558.4, [M+H]⁺, ESI pos.

### Step 8:2-[4,7-Dichloro-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide

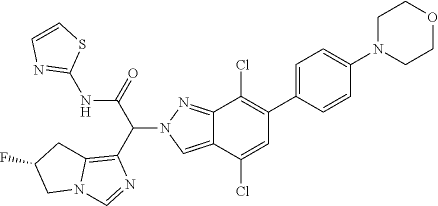 [SMILES: O=C(Nc1nccs1)C(c1ncn2c1C[C@@H](F)C2)n1cc2c(Cl)cc(-c3ccc(N4CCOCC4)cc3)c(Cl)c2n1]

To a solution of ethyl 2-[4,7-dichloro-6-(4-morpholinophenyl)indazol-2-yl]-2-[(6R)-6-fluoro-6,7-dihydro-5H-pyrrolo[1,2-c]imidazol-1-yl]acetate (Example 1, step 7) (40 mg, 0.071 mmol) in tetrahydrofuran (1.1 ml) were added LiOH 1M (101 μl, 0.10 mmol, 1.5 equiv.) and water (400 μl). The mixture was stirred for 30 minutes at room temperature. The mixture was concentrated and dried. The residue was dissolved in N,N-dimethylformamide (1.1 ml). After addition of thiazol-2-amine (9 mg, 0.086 mmol, 1.2 equiv.), HATU (33 mg, 0.086 mmol, 1.2 equiv.) and Hunig's base (28 mg, 0.037 ml, 0.21 mmol, 3 equiv.) the mixture was stirred for 1 hour at room temperature. Water was added and the mixture was extracted with ethyl acetate. The organic layers were combined, dried with sodium sulfate, filtered and concentrated. The crude material was purified by flash chromatography (SiO₂, 0% to 40% methanol in ethyl acetate) to give the title compound (22 mg, 50% yield) as light brown solid. m/z 612.4, [M+H]⁺, ESI pos.

### Example 2

### 2-[4-(Difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide

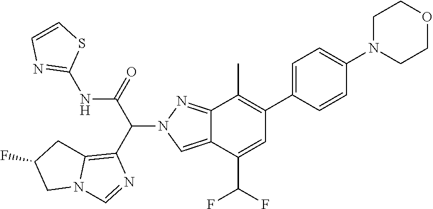 [SMILES: Cc1c(-c2ccc(N3CCOCC3)cc2)cc(C(F)F)c2cn(C(C(=O)Nc3nccs3)c3ncn4c3C[C@@H](F)C4)nc12]

### Step 1: Ethyl 2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]acetate

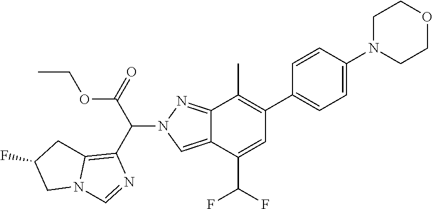 [SMILES: CCOC(=O)C(c1ncn2c1C[C@@H](F)C2)n1cc2c(C(F)F)cc(-c3ccc(N4CCOCC4)cc3)c(C)c2n1]

A suspension of Intermediate 1 (0.5 g, 1.06 mmol, Eq: 1.0), (4-morpholinophenyl) boronic acid (CAS 186498-02-2, 329 mg, 1.59 mmol, Eq: 1.5) and K₂CO₃ (183 mg, 1.33 mmol, Eq: 1.25) in 2-MeTHF (6 mL), water (1 mL) was degassed with argon for 10 min. Dichloro[bis(diphenylphosphinophenyl)ether]palladium (II) (CAS 205319-06-8, 91 mg, 127 μmol, Eq: 0.12) was added. The reaction mixture was stirred for 5 hours at 85° C. AcOH (191 mg, 182 μL, 3.18 mmol, Eq: 3.0) was added. The reaction mixture was poured into EtOAc/THF 2:1 and washed with water and brine. The organic layer was dried over Na₂SO₄ and concentrated in vacuo. The crude material was purified by flash chromatography (silica gel, 12 g, 0% to 50% (EtOAc/EtOH/aq. NH₃ 75:25:2) in heptane) to give the title compound as an off-white solid (433 mg, 743 μmol, 70% yield). m/z 554.4 [M+H]⁺, ESI pos.

### Step 2:2-[4-(Difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide

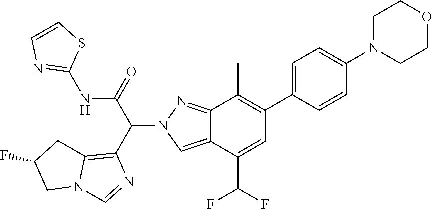 [SMILES: Cc1c(-c2ccc(N3CCOCC3)cc2)cc(C(F)F)c2cn(C(C(=O)Nc3nccs3)c3ncn4c3C[C@@H](F)C4)nc12]

To a solution of ethyl 2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]acetate (720 mg, 1.3 mmol, Eq: 1.0) in THF (9 mL) and MeOH (9 mL) was added LiOH (1 M aq., 2.6 mL, 2.6 mmol, Eq: 2.0). The reaction mixture was stirred for 2 hours at room temperature. HCl (5 N aq., 520 μL, 2.6 mmol, Eq: 2.0) was added (pH 6). Toluene was added, the reaction mixture was concentrated in vacuo. The carboxylic acid was dissolved in DMSO (6 mL), thiazol-2-amine (195 mg, 1.95 mmol, Eq: 1.5), DIPEA (840 mg, 1.14 mL, 6.5 mmol, Eq: 5.0) and HATU (742 mg, 1.95 mmol, Eq: 1.5) were added. The reaction mixture was stirred for 1.5 hours at room temperature. The reaction mixture was poured into EtOAc/THF 2:1 and washed with water and brine. The organic layer was dried over Na₂SO₄ and concentrated in vacuo. The crude material was purified by flash chromatography (silica gel, 40 g, 0% to 5% MeOH in DCM). The product was freeze-dried to give the title compound as an off-white solid (501 mg, 63% yield). m/z 608.3 [M+H]⁺, ESI pos.

### Example 3

### 2-[4-(Difluoromethyl)-7-methyl-6-(4-morpholinophenyl)indazol-2-yl]-2-spiro[6,7-dihydropyrrol[1,2-c]imidazole-5,1′-cyclopropane]-1-yl-N-thiazol-2-yl-acetamide

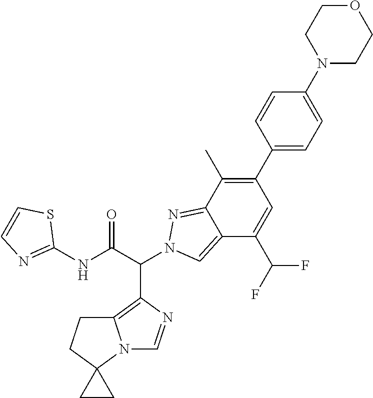 [SMILES: Cc1c(-c2ccc(N3CCOCC3)cc2)cc(C(F)F)c2cn(C(C(=O)Nc3nccs3)c3ncn4c3CCC43CC3)nc12]

### Step 1:1-Bromo-5-(difluoromethyl)-3-fluoro-2-methyl-benzene

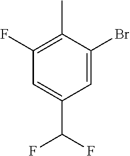

To a cooled solution of 3-bromo-5-fluoro-4-methylbenzaldehyde (CAS #1370411-47-4, 20.5 g, 89.7 mmol, 1.0 equiv) in dichloromethane (98 mL) was added morpholinosulfur trifluoride (CAS #51010-74-3, 24.8 g, 17.3 mL, 135 mmol, 1.5 equiv) in portions. The reaction mixture was stirred at 0-5° C. for 20 minutes, then at room temperature for 16 hours. Saturated aqueous NaHCO₃-solution (300 mL) was added carefully under ice cooling. The reaction mixture was stirred at room temperature for 1 hour. The reaction mixture was poured into dichloromethane and washed with water. The organic layer was dried over sodium sulfate, filtered and concentrated in vacuo. The crude material was purified by flash chromatography (silica gel, 120 g, 100% pentane) to afford the title compound (18.6 g, 87% yield) as a colourless oil. ¹H NMR (300 MHZ, chloroform-d) δ=7.50 (s, 1H), 7.16 (d, J=9.1 Hz, 1H), 6.57 (t, J=56.0 Hz, 1H), 2.50-2.22 (m, 3H)

### Step 2:6-Bromo-4-(difluoromethyl)-7-methyl-1H-indazole

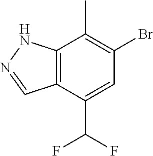

A solution of 1-bromo-5-(difluoromethyl)-3-fluoro-2-methyl-benzene (Example 1, step 1) (26.4 g, 110 mmol, 1.0 equiv) in tetrahydrofuran (240 mL) was cooled to −75° C. Lithium diisopropylamide solution (2 M in tetrahydrofuran/heptane/ethylbenzene, 66.3 mL, 133 mmol, 1.2 equiv) was added dropwise maintaining the temperature below −70° C. The reaction mixture was stirred at −75° C. for 30 minutes. Ethyl formate (16.4 g, 17.7 mL, 220 mmol, 2.0 equiv) was added below −70° C. The reaction mixture was stirred at −75° C. for 30 minutes. Acetic acid (16.6 g, 15.8 mL, 277 mmol, 2.5 equiv) was added below −55° C. The reaction mixture was allowed to warm up to room temperature, poured into ethyl acetate and washed with diluted aqueous HCl-solution, water and brine. The organic layer was dried over sodium sulfate and concentrated in vacuo to give presumed 4-bromo-6-(difluoromethyl)-2-fluoro-3-methyl-benzaldehyde as a yellow oil (29.5 g) which was used without further purification. The crude presumed 4-bromo-6-(difluoromethyl)-2-fluoro-3-methyl-benzaldehyde (29.5 g) was dissolved in dimethoxyethane (150 mL). O-Methylhydroxylamine hydrochloride (10.2 g, 122 mmol, 1.1 equiv) and potassium carbonate (30.6 g, 221 mmol, 2.0 equiv) were added. The reaction mixture was stirred at 45° C. for 2.5 hours then filtered through sintered glass and washed with dimethoxyethane (2×). The filtrate was concentrated in vacuo. The oxime ether intermediate was dissolved in dimethylsulfoxide (150 mL). Hydrazine hydrate (83 g, 80.5 mL, 1.66 mol, 15 equiv) was added. The reaction mixture was stirred at 110° C. for for 3 hours. The reaction mixture was poured into a mixture of ethyl acetate/tetrahydrofuran 5:1. The organic layer was washed with water and brine, dried over sodium sulfate and concentrated in vacuo. The crude material was purified by flash chromatography (silica gel, 2×120 g, gradient 0% to 30% ethyl acetate in heptane) to afford the title compound (13.5 g, 45% yield) as a white solid (13.5 g, 45% yield). LCMS: m/z 260.9/262.8 [M+H]⁺, ESI pos, Br isotopes.

### Step 3: Ethyl 2-[6-bromo-4-(difluoromethyl)-7-methyl-indazol-2-yl]acetate

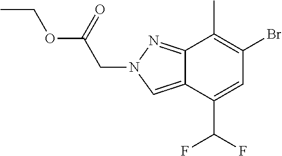 [SMILES: CCOC(=O)Cn1cc2c(C(F)F)cc(Br)c(C)c2n1]

To a solution of 6-bromo-4-(difluoromethyl)-7-methyl-1H-indazole (Example 1, step 2) (19 g, 72.8 mmol, 1.0 equiv) in N,N-dimethylformamide (75 mL) was added ethyl 2-bromoacetate (CAS #105-36-2, 18.2 g, 12.2 mL, 109 mmol, 1.5 equiv). The reaction mixture was stirred at 100° C. for 16 hours. The reaction mixture was poured into ethyl acetate and washed with water and brine.

The organic layer was dried over sodium sulfate, filtered and concentrated in vacuo. The crude material was purified by flash chromatography (silica gel, 2×120 g, gradient 0% to 20% ethyl acetate in heptane) to afford the title compound (21.2 g, 80% yield) as a yellow solid. LCMS: m/z 346.9/348.8 [M+H]⁺, ESI pos, Br isotopes.

### Step 4: tert-Butyl (5R)-5-[2-[6-bromo-4-(difluoromethyl)-7-methyl-indazol-2-yl]-3-ethoxy-3-oxo-propanoyl]-4-azaspiro[2.4]heptane-4-carboxylate

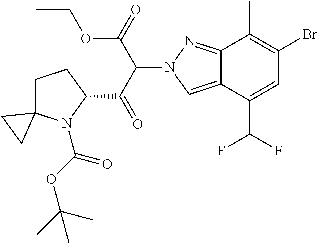 [SMILES: CCOC(=O)C(C(=O)[C@H]1CCC2(CC2)N1C(=O)OC(C)(C)C)n1cc2c(C(F)F)cc(Br)c(C)c2n1]

### Preparation of tert-butyl (5R)-5-(imidazole-1-carbonyl)-4-azaspiro[2.4]heptane-4-carboxylate

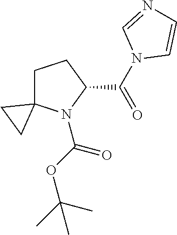 [SMILES: CC(C)(C)OC(=O)N1[C@@H](C(=O)n2ccnc2)CCC12CC2]

To a solution of (5R)-4-tert-butoxycarbonyl-4-azaspiro[2.4]heptane-5-carboxylic acid (CAS #2007916-06-3, 1.02 g, 4.21 mmol, 1.0 equiv) in dichloromethane (17 mL) was added 1,1′-carbonyldiimidazole (818 mg, 5.04 mmol, 1.2 eq) in three portions and the reaction mixture was stirred at room temperature for 2.5 hours. The reaction mixture was poured into saturated aqueous NaHCO₃-solution and extracted three times with dichloromethane. The combined organic layers were dried over sodium sulfate, filtered and concentrated in vacuo to afford tert-butyl (5R)-5-(imidazole-1-carbonyl)-4-azaspiro[2.4]heptane-4-carboxylate (1.30 g, 95% yield, 90% purity) as an off-white solid, which was used directly in the next step.

A solution of ethyl 2-[6-bromo-4-(difluoromethyl)-7-methyl-indazol-2-yl]acetate (Example 1, step 3) (1.2 g, 3.46 mmol, 1.0 equiv) in tetrahydrofuran (16 mL) was cooled to −50° C. NaHMDS (1 M in tetrahydrofuran) (4 mL, 4 mmol, 1.16 equiv) was added and the reaction mixture was stirred at −50° C. for 45 minutes. A solution of aforementioned tert-butyl (5R)-5-(imidazole-1-carbonyl)-4-azaspiro[2.4]heptane-4-carboxylate (1.29 g, 3.98 mmol, 1.15 equiv) in tetrahydrofuran (16 mL) was added dropwise at −50° C. The reaction mixture was stirred at −50° C. for 30 minutes. Then, the cooling bath was removed and the reaction mixture was allowed to warm up to room temperature. The reaction mixture was stirred at room temperature for 2 hours. The reaction mixture was cooled and quenched with saturated aqueous NH₄Cl-solution, diluted with water and extracted with ethyl acetate. The aqueous layer was backextracted with ethyl acetate. The organic layers were washed with saturated aqueous NaHCO₃-solution then brine. The combined organic layers were dried over sodium sulfate, filtered and concentrated in vacuo to afford the title compound (2.37 g, 96% yield, 80% purity) as a light brown foam, which was used without further purification. LCMS: m/z 570.3/572.3 [M+H]⁺, ESI pos, Br isotopes.

### Step 5: Ethyl 2-[6-bromo-4-(difluoromethyl)-7-methyl-indazol-2-yl]-2-spiro[6,7-dihydropyrrolo[1,2-c]imidazole-5,1′-cyclopropane]-1-yl-acetate

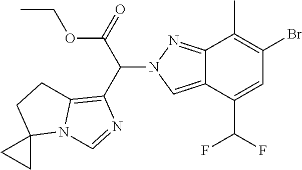 [SMILES: CCOC(=O)C(c1ncn2c1CCC21CC1)n1cc2c(C(F)F)cc(Br)c(C)c2n1]

A mixture of tert-butyl (5R)-5-[2-[6-bromo-4-(difluoromethyl)-7-methyl-indazol-2-yl]-3-ethoxy-3-oxo-propanoyl]-4-azaspiro[2.4]heptane-4-carboxylate (Example 1, step 4) (2.36 g, 3.31 mmol, 1.0 equiv, 80% purity) and HCl (4 M in 1,4-dioxane, 4.2 mL, 16.8 mmol, 5.07 equiv). The reaction mixture was stirred at room temperature for 1 hour. HCl (4 M in 1,4-dioxane, 1 mL, 4.0 mmol, 1.21 equiv) was added and the reaction mixture was stirred at room temperature for 30 minutes. HCl (4 M in 1,4-dioxane, 1 mL, 4.0 mmol, 1.21 equiv.) was added and the reaction mixture was stirred at room temperature for 30 minutes. The reaction mixture was diluted with ethanol (7.6 mL) and water (2.0 mL). Potassium thiocyanate (419 mg, 4.31 mmol, 1.3 equiv) was added and the reaction mixture was stirred at room temperature for 1 hour. The reaction mixture was cooled and pyridine (3.91 g, 4 mL, 49.46 mmol, 14.9 equiv) was added slowly. The reaction mixture was stirred at room temperature for 1 hour. The reaction mixture was poured into 1 N KHSO₄+water and extracted three times with ethyl acetate. The organic layers were washed with brine, dried over sodium sulfate, filtered and concentrated in vacuo. The crude material was dissolved in acetic acid (7.6 mL) and cooled to 0° C. Hydrogen peroxide (35 wt. % solution in water, 0.580 mL, 6.62 mmol, 2.0 equiv) was added dropwise at 0° C. The reaction mixture was stirred at room temperature for 1 hour. The excess of hydrogen peroxide was destroyed with a 1 M aqueous solution of Na₂S₂O₃. The mixture was carefully basified with solid Na₂CO₃ and extracted twice with ethyl acetate. The organic layers were washed with brine, combined, dried over sodium sulfate, filtered and concentrated in vacuo. The crude material was adsorbed on ISOLUTE HM-N and purified by flash chromatography (silica gel, 24 g, gradient 0% to 100% ethyl acetate in heptane). All fractions containing product were combined and concentrated in vacuo to afford the title compound (882 mg, 53% yield) as a light yellow solid. LCMS: m/z 479.3/481.3 [M+H]⁺, ESI pos, Br isotopes.

### Step 6: Ethyl 2-[4-(difluoromethyl)-7-methyl-6-(4-morpholinophenyl)indazol-2-yl]-2-spiro[6,7-dihydropyrrolo[1,2-c]imidazole-5,1′-cyclopropane]-1-yl-acetate

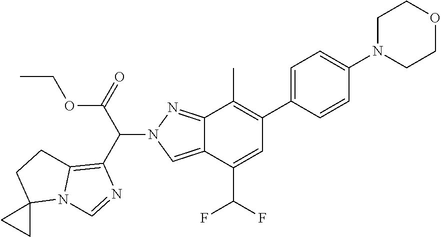 [SMILES: CCOC(=O)C(c1ncn2c1CCC21CC1)n1cc2c(C(F)F)cc(-c3ccc(N4CCOCC4)cc3)c(C)c2n1]

A mixture of ethyl 2-[6-bromo-4-(difluoromethyl)-7-methyl-indazol-2-yl]-2-spiro[6,7-dihydropyrrolo[1,2-c]imidazole-5,1′-cyclopropane]-1-yl-acetate (Example 1, step 5) (350 mg, 0.69 mmol, 1.00 equiv), (4-morpholinophenyl) boronic acid (CAS #186498-02-2, 187 mg, 0.90 mmol, 1.30 equiv), cesium carbonate (679 mg, 2.08 mmol, 3.00 equiv) and [1,1′-bis(diphenylphosphino) ferrocene]dichloropalladium (II) complex with dichloromethane (82 mg, 0.10 mmol, 0.14 equiv) in 1,4-dioxane (6.0 mL) was flushed with argon and stirred at 100° C. for 2 hours. The reaction mixture was cooled to room temperature and then extracted with ethyl acetate and water. The aqueous layer was backextracted with ethyl acetate. The organic layers were washed with water and brine. The combined organic layers were dried over sodium sulfate, filtered and concentrated in vacuo. The crude material was adsorbed on ISOLUTE HM-N and purified by flash chromatography (silica gel, 24 g, gradient 0% to 90% ethyl acetate in heptane). All fractions containing product were combined and concentrated to afford the title compound (352 mg, 81% yield, 90% purity) as an off-white foam. LCMS: m/z 562.5 [M+H]⁺, ESI pos.

### Step 7:2-[4-(Difluoromethyl)-7-methyl-6-(4-morpholinophenyl)indazol-2-yl]-2-spiro[6,7-dihydropyrrolo[1,2-c]imidazole-5,1′-cyclopropane]-1-yl-N-thiazol-2-yl-acetamide

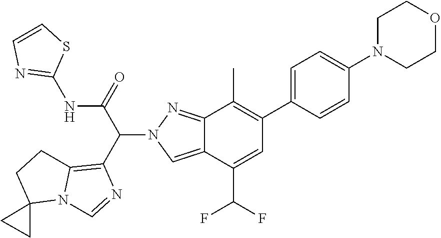 [SMILES: Cc1c(-c2ccc(N3CCOCC3)cc2)cc(C(F)F)c2cn(C(C(=O)Nc3nccs3)c3ncn4c3CCC43CC3)nc12]

To a solution of ethyl 2-[4-(difluoromethyl)-7-methyl-6-(4-morpholinophenyl)indazol-2-yl]-2-spiro[6,7-dihydropyrrolo[1,2-c]imidazole-5,1′-cyclopropane]-1-yl-acetate (Example 1, step 6) (346 mg, 0.55 mmol, 1.0 equiv) in ethanol (1.8 mL) and tetrahydrofuran (1.8 mL) was added lithium hydroxide (1 M aqueous solution, 0.64 mL, 0.64 mmol, 1.15 equiv). The reaction mixture was stirred at room temperature for 1.5 hours. The reaction mixture was evaporated and co-evaporated with toluene twice. The residue was suspended in N,N-dimethylformamide (1.8 mL) and N,N-diisopropylethylamine (0.30 mL, 1.72 mmol, 3.1 equiv), thiazol-2-amine (72 mg, 0.72 mmol, 1.3 equiv) and HATU (274 mg, 0.72 mmol, 1.3 equiv) were added. The reaction mixture was stirred at room temperature for 45 minutes. The reaction mixture was extracted with ethyl acetate and water. The aqueous layer was backextracted with ethyl acetate. The organic layers were washed three times with water and once with brine. The combined organic layers were dried over sodium sulfate, filtered and concentrated in vacuo. The crude product was adsorbed on ISOLUTE HM-N and purified by flash chromatography (silica gel, 25 g, gradient 0% to 5% methanol in dichloromethane). All fractions containing product were combined and concentrated in vacuo. The residue was adsorbed on ISOLUTE HM-N and repurified by flash chromatography (Si-amine, 12 g, gradient 0% to 10% methanol in ethyl acetate). All fractions containing product were combined and concentrated to afford the title compound (183 mg, 53% yield) as an off-white foam. LCMS: m/z 616.4 [M+H]⁺, ESI pos.

### Example 4

### 2-(6,7-Dihydro-5H-pyrrolo[1,2-c]imidazol-1-yl)-2-(4-fluoro-6-((4-((4-hydroxypiperidin-1-yl)methyl)phenyl)ethynyl)-2H-indazol-2-yl)-N-(thiazol-2-yl)acetamide

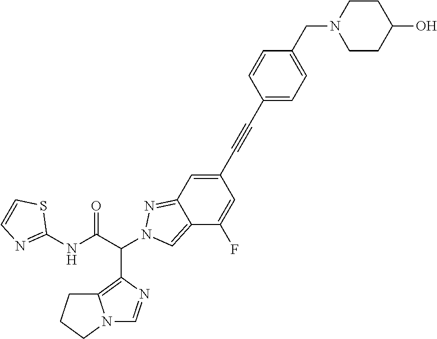 [SMILES: O=C(Nc1nccs1)C(c1ncn2c1CCC2)n1cc2c(F)cc(C#Cc3ccc(CN4CCC(O)CC4)cc3)cc2n1]

### Step 1: Ethyl 2-(6-bromo-4-fluoro-indazol-2-yl)acetate

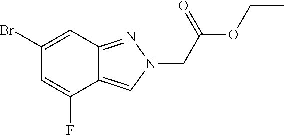 [SMILES: CCOC(=O)Cn1cc2c(F)cc(Br)cc2n1]

A mixture of 6-bromo-4-fluoro-1H-indazole (CAS 885520-23-0) (1 equiv), ethyl 2-bromoacetate (2 equiv) and N,N-dimethylacetamide (small amount to produce a solution) was heated to 100° C. until completion of the reaction. After cooling to room temperature, ice was added and the precipitated solid was collected by filtration and washed with water. The title compound (off-white solid, 53% yield) was purified using flash chromatography (silica gel, 0% to 40% ethyl acetate in n-heptane). ¹H NMR (chloroform-d, 300 MHz) δ=8.08 (d, 1H, J=0.8 Hz), 7.68 (t, 1H, J=1.1 Hz), 6.87 (dd, 1H, J=1.3, 9.6 Hz), 5.18 (s, 2H), 4.28 (q, 2H, J=7.3 Hz), 1.30 (t, 3H, J=7.2 Hz). MS (ESI) m/z 302.9 [M+H]⁺.

### Step 2: 2[-2-(6-Bromo-4-fluoro-indazol-2-yl)-3-ethoxy-3-oxo-propanoyl]pyrrolidine-1-carboxylate

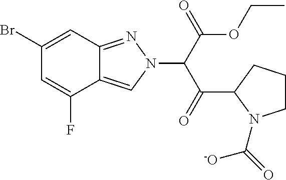

Ethyl 2-(6-bromo-4-fluoro-indazol-2-yl)acetate (1 eq.) was reacted with a CDI pre-activated tert-butoxycarbonyl)-L-proline (CAS15761-39-4) (1.55 eq.) in the presence of LDA (1.55 eq.) in THF at −70° C. to rt. This reaction yielded the title compound 2 [-2-(6-bromo-4-fluoro-indazol-2-yl)-3-ethoxy-3-oxo-propanoyl]pyrrolidine-1-carboxylate.

### Step 3: ethyl 2-(6-bromo-4-fluoro-indazol-2-yl)-2-(3-thioxo-2,5,6,7-tetrahydropyrrolo[1,2-c]imidazole-1-yl)acetate

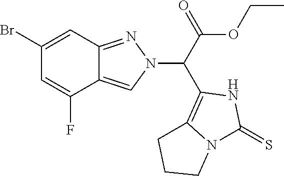 [SMILES: CCOC(=O)C(c1[nH]c(=S)n2c1CCC2)n1cc2c(F)cc(Br)cc2n1]

2 [-2-(6-Bromo-4-fluoro-indazol-2-yl)-3-ethoxy-3-oxo-propanoyl]pyrrolidine-1-carboxylate (1 eq) was reacted with HCl (in dioxane) at rt. Nest, KSCN (1.33 eq.) was added in the presence of HCl in ethanol at rt. This reaction yielded ethyl 2-(6-bromo-4-fluoro-indazol-2-yl)-2-(6,7-dihydro-5H-pyrrolo[1,2-c]imidazol-1-yl)acetate.

### Step 4: Ethyl 2-(6-bromo-4-fluoro-indazol-2-yl)-2-(6,7-dihydro-5H-pyrrolo[1,2-c]imidazol-1-yl)acetate

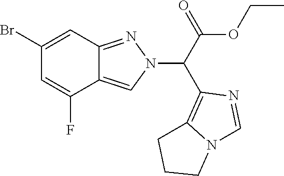 [SMILES: CCOC(=O)C(c1ncn2c1CCC2)n1cc2c(F)cc(Br)cc2n1]

To a solution of ethyl 2-(6-bromo-4-fluoro-indazol-2-yl)-2-(6,7-dihydro-5H-pyrrolo[1,2-c]imidazol-1-yl)acetate (2.88 mmol, 1 equiv) in acetic acid (10 mL) was added dropwise hydrogen peroxide, 35% (1.12 g, 1.01 mL, 11.5 mmol, 4 equiv) at 10° C. The reaction mixture was stirred for 1 hour at room temperature. The excess of hydrogen peroxide was destroyed by addition of saturated sodium sulfit solution. After addition of some water (just enough to dissolve all salts) and ethyl acetate, the mixture was brought to pH 9 by careful addition of solid sodium carbonate. The mixture was extracted with ethyl acetate. The organic layers were washed with water, dried over sodium sulfate and concentrated. The product was purified by chromatography (silica gel, 0-100% ethyl acetate in n-heptane) to give the title compound as brown gum (51% yield over steps 2 to 4). 1H NMR (300 MHz, chloroform-d) δ=8.22 (s, 1H), 7.66 (t, J=1.0 Hz, 1H), 7.50 (s, 1H), 6.80 (dd, J=1.2, 9.5 Hz, 1H), 6.39 (s, 1H), 4.39-4.19 (m, 2H), 4.10-3.97 (m, 2H), 2.95-2.60 (m, 4H), 1.42-1.10 (m, 3H). MS (ESI) m/z 409.0 [M+H]⁺.

### Step 5:2-(6-Bromo-4-fluoro-indazol-2-yl)-2-(6,7-dihydro-5H-pyrrolo[1,2-c]imidazol-1-yl)-N-thiazol-2-yl-acetamide

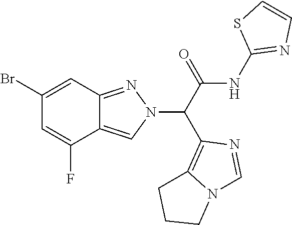 [SMILES: O=C(Nc1nccs1)C(c1ncn2c1CCC2)n1cc2c(F)cc(Br)cc2n1]

To a solution of ethyl 2-(6-bromo-4-fluoro-indazol-2-yl)-2-(6,7-dihydro-5H-pyrrolo[1,2-c]imidazol-1-yl)acetate (1 eq) in ethanol (3 mL) was added lithium hydroxide 1M (1.1 eq.). The reaction was stirred for 1 hour, the solvent was evaporated and the residue was concentrated and coevaporated with toluene twice to remove water. The reaction mixture was diluted in N,N-dimethylformamide (3 mL) and N,N-diisopropylethylamine (345 mg, 0.466 mL, 2.67 mmol, 3 equiv), thiazol-2-ylamine (116 mg, 1.16 mmol, 1.3 equiv) and HATU (440 mg, 1.16 mmol, 1.3 equiv) were added. After stirring at room temperature for 1 hour, the reaction was diluted with water and extracted twice with ethyl acetate. The combined organic layers were dried over sodium sulfate, filtered and concentrated. The residue was purified by flash chromatography (silica gel, methanol in dichloromethane 0% to 10%) to give 2-(6-bromo-4-fluoro-indazol-2-yl)-2-(6,7-dihydro-5H-pyrrolo[1,2-c]imidazol-1-yl)-N-thiazol-2-yl-acetamide.

### Step 6:2-(6,7-Dihydro-5H-pyrrolo[1,2-c]imidazol-1-yl)-2-(4-fluoro-6-((4-((4-hydroxypiperidin-1-yl)methyl)phenyl)ethynyl)-2H-indazol-2-yl)-N-(thiazol-2-yl)acetamide

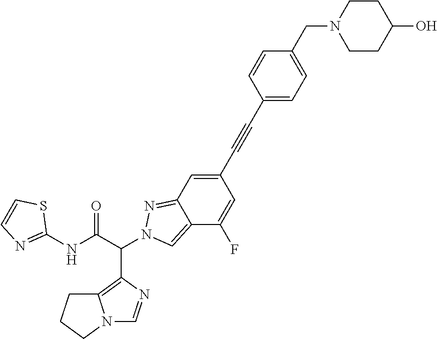 [SMILES: O=C(Nc1nccs1)C(c1ncn2c1CCC2)n1cc2c(F)cc(C#Cc3ccc(CN4CCC(O)CC4)cc3)cc2n1]

To a solution of 2-(6-bromo-4-fluoro-indazol-2-yl)-2-(6,7-dihydro-5H-pyrrolo[1,2-c]imidazol-1-yl)-N-thiazol-2-yl-acetamide (1 eq.) in N,N-dimethylformamide (5 mL) were added added 4-ethynylbenzaldehyde (49 mg, 0.375 mmol, 1 equiv), triethylamine (113.83 mg, 0.157 mL, 1.12 mmol, 3 equiv), triphenylphosphine (10 mg, 0.037 mmol, 0.100 equiv), bis(triphenylphosphine) palladium (II) chloride (13 mg, 0.019 mmol, 0.050 equiv) and copper (I) iodide (3.57 mg, 0.019 mmol, 0.050 equiv). The vial was capped and heated in the microwave at 110° C. for 20 minutes then 4-ethynylbenzaldehyde (49 mg, 0.375 mmol, 1 equiv) was added and the vial was again heated at 110° C. for 20 minutes. This procedure was repeated three times. In total, the reaction mixture was stirred 5×20 minutes at 110° C. The reaction mixture was poured into water and extracted with ethyl acetate (4×). The combined organic layers were washed with brine, dried over sodium sulfate, filtered and concentrated in vacuo. The crude was purified by flash chromatography (silica gel, methanol in ethyl acetate 0% to 10%) and was further combined with piperidin-4-ol (1.3 eq) and sodium triacetoxyborohydride (70 mg, 0.332 mmol, 1.6 equiv). The reaction mixture was stirred at room temperature for 3 hours. The reaction mixture was poured into NaHCO₃ sat sol and extracted three times with a mixture of dichloromethane/methanol 9:1. The combined organic layers were dried over sodium sulfate, filtered and concentrated in vacuo. The crude was purified by flash chromatography (silica gel, methanol in ethyl acetate 0% to 10%) to give the title compound (16% yield, off-white solid).

¹H NMR (300 MHZ, chloroform-d) δ=8.26 (s, 1H), 7.71 (s, 1H), 7.58 (s, 1H), 7.54-7.46 (m, 3H), 7.34-7.29 (m, 2H), 7.00 (d, J=3.6 Hz, 1H), 6.87-6.79 (m, 1H), 6.52 (s, 1H), 4.06-3.96 (m, 2H), 3.77-3.66 (m, 1H), 3.55-3.47 (m, 2H), 2.79-2.70 (m, 2H), 2.65-2.51 (m, 4H), 2.23-2.10 (m, 2H), 1.94-1.85 (m, 2H), 1.72-1.47 (m, 4H). MS (ESI) m/z 594.6 [M−H]⁻.

### Example 5

### 2-[4-(Difluoromethyl)-6-[2-[4-[[4-(hydroxymethyl)-1-piperidyl]methyl]phenyl]ethynyl]-7-methyl-indazol-2-yl]-2-(6,7-dihydro-5H-pyrrolo[1,2-c]imidazol-1-yl)-N-thiazol-2-yl-acetamide

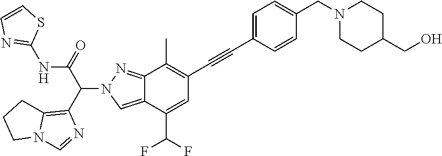 [SMILES: Cc1c(C#Cc2ccc(CN3CCC(CO)CC3)cc2)cc(C(F)F)c2cn(C(C(=O)Nc3nccs3)c3ncn4c3CCC4)nc12]

### Step 1: tert-Butyl (2R)-2-[2-[6-bromo-4-(difluoromethyl)-7-methylindazol-2-yl]-3-ethoxy-3-oxopropanoyl]pyrrolidine-1-carboxylate

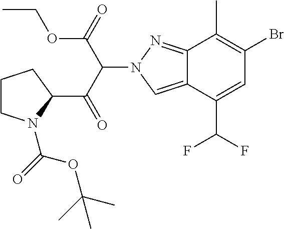 [SMILES: CCOC(=O)C(C(=O)[C@@H]1CCCN1C(=O)OC(C)(C)C)n1cc2c(C(F)F)cc(Br)c(C)c2n1]

Ethyl 2-[6-bromo-4-(difluoromethyl)-7-methyl-indazol-2-yl]acetate (0.65 g, 1.87 mmol, Eq: 1.0) was dissolved in THF (7.58 mL) and cooled to −75° C. LDA (2 M in THF, 1.12 mL, 2.25 mmol, Eq: 1.20) was added dropwise within 5 min. The reaction mixture was stirred for 40 min at −75° C. A solution of tert-butyl (2S)-2-(imidazole-1-carbonyl) pyrrolidine-1- carboxylate (prepared from (2S)-1-tert-butoxycarbonyl-4-fluoro-pyrrolidine-2-carboxylic acid) (0.77 g, 2.9 mmol, Eq: 1.55) in THF (7.58 mL) was added slowly at −75° C., stirred for 30 min at −75° C. then allowed to warm up to room temperature and stirred for 18 hours at room temperature. After the addition of saturated aqueous NH₄Cl, the reaction mixture was extracted twice with EtOAc. The organic layers were washed with water. The combined organic layers were dried over Na₂SO₄ and concentrated in vacuo to give the title compound (1.41 g, 72% purity, 99%, yield) which was used in the next step without further purification. m/z 544.1, 546.0 [M+H]⁺, ESI pos. Br isotopes.

### Step 2: Ethyl 2-[6-bromo-4-(difluoromethyl)-7-methyl-indazol-2-yl]-2-(3-thioxo-2,5,6,7-tetrahydropyrrolo[1,2-c]imidazol-1-yl)acetate

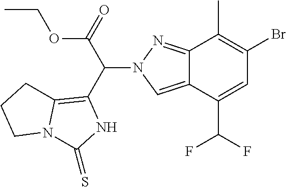 [SMILES: CCOC(=O)C(c1[nH]c(=S)n2c1CCC2)n1cc2c(C(F)F)cc(Br)c(C)c2n1]

By analogy with Example 7, Step 5, tert-butyl (2R)-2-[2-[6-bromo-4-(difluoromethyl)-7-methylindazol-2-yl]-3-ethoxy-3-oxopropanoyl]pyrrolidine-1-carboxylate (1.4 g, 72% purity, 1.85 mmol) was treated with HCl 4 M in dioxane and potassium thiocyanate to give the title compound as a brown oil (1.07 g, 85% purity, 100% yield) which was used in the next step without further purification. m/z 485.0, 486.9 [M+H]⁺, ESI pos, Br isotopes.

### Step 3: Ethyl 2-[6-bromo-4-(difluoromethyl)-7-methyl-indazol-2-yl]-2-(6,7-dihydro-5H-pyrrolo[1,2-c]imidazol-1-yl)acetate

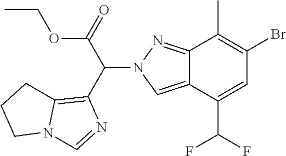 [SMILES: CCOC(=O)C(c1ncn2c1CCC2)n1cc2c(C(F)F)cc(Br)c(C)c2n1]

By analogy with Example 7, Step 6, ethyl 2-[6-bromo-4-(difluoromethyl)-7-methyl-indazol-2-yl]-2-(3-thioxo-2,5,6,7-tetrahydropyrrolo[1,2-c]imidazol-1-yl)acetate (1.06 g, 85% purity, 1.86 mmol) was treated with hydrogen peroxide and p-toluenesulfonic acid monohydrate to give the title compound as a yellow foam (360 mg, 43% yield). m/z 453.0, 454.9 [M+H]⁺, ESI pos, Br isotopes.

### Step 4:2-[6-Bromo-4-(difluoromethyl)-7-methyl-indazol-2-yl]-2-(6,7-dihydro-5H-pyrrolo[1,2-c]imidazol-1-yl)-N-thiazol-2-yl-acetamide

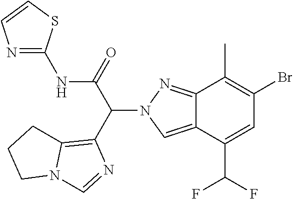 [SMILES: Cc1c(Br)cc(C(F)F)c2cn(C(C(=O)Nc3nccs3)c3ncn4c3CCC4)nc12]

The title compound was obtained as a light yellow foam, MS: m/e=509.1/511.1 (M+H⁺), using chemistry similar to that described in Example 7, step 7 starting from ethyl 2-[6-bromo-4-(difluoromethyl)-7-methyl-indazol-2-yl]-2-(6,7-dihydro-5H-pyrrolo[1,2-c]imidazol-1-yl)acetate (Example 5, step 3) and thiazol-2-amine.

### Step 5:2-[4-(Difluoromethyl)-6-[2-[4-[[4-(hydroxymethyl)-1-piperidyl]methyl]phenyl]ethynyl]-7-methyl-indazol-2-yl]-2-(6,7-dihydro-5H-pyrrolo[1,2-c]imidazol-1-yl)-N-thiazol-2-yl-acetamide

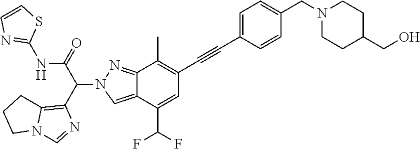 [SMILES: Cc1c(C#Cc2ccc(CN3CCC(CO)CC3)cc2)cc(C(F)F)c2cn(C(C(=O)Nc3nccs3)c3ncn4c3CCC4)nc12]

The title compound was obtained as a brown solid, MS: m/e=656.5 (M+H⁺), using chemistry similar to that described in Example 7, step 10 starting from 2-[6-bromo-4-(difluoromethyl)-7-methyl-indazol-2-yl]-2-(6,7-dihydro-5H-pyrrolo[1,2-c]imidazol-1-yl)-N-thiazol-2-yl-acetamide (Example 5, step 4) and [1-[(4-ethynylphenyl)methyl]-4-piperidyl]methanol hydrochloride (Example 7, step 9).

### Example 6

### 2-[4-(Difluoromethyl)-6-[2-[4-[[4-(hydroxymethyl)-1-piperidyl]methyl]phenyl]ethynyl]-7-methyl-indazol-2-yl]-2-(5-ethyl-5-methyl-6,7-dihydropyrrolo[1,2-c]imidazol-1-yl)-N-thiazol-2-yl-acetamide

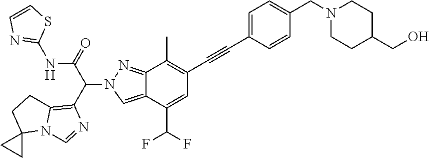 [SMILES: Cc1c(C#Cc2ccc(CN3CCC(CO)CC3)cc2)cc(C(F)F)c2cn(C(C(=O)Nc3nccs3)c3ncn4c3CCC43CC3)nc12]

### Step 1: (5S)-5-[2-[6-Bromo-4-(difluoromethyl)-7-methyl-indazol-2-yl]-3-ethoxy-3-keto-propanoyl]-4-azaspiro[2.4]heptane-4-carboxylic acid tert-butyl ester

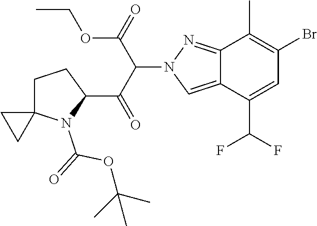 [SMILES: CCOC(=O)C(C(=O)[C@@H]1CCC2(CC2)N1C(=O)OC(C)(C)C)n1cc2c(C(F)F)cc(Br)c(C)c2n1]

In analogy to Example 1, step 4, (5S)-4-tert-butoxycarbonyl-4-azaspiro[2.4]heptane-5-carboxylic acid was treated with carbonyldiimidazole to give solution A. 2-[6-Bromo-4-(difluoromethyl)-7-methyl-indazol-2-yl]acetic acid ethyl ester was deprotonated with LDA and treated with solution A at −78° C. After stirring at room temperature overnight and workup in analogy to Example 7, step 4, the crude title compound was obtained as a light yellow foam and used for the next step without further purification. MS: m/e=572.3 ([M+H]⁺)

### Step 2:2-[6-Bromo-4-(difluoromethyl)-7-methyl-indazol-2-yl]-2-spiro[6,7-dihydropyrrol[1,2-c]imidazole-5,1′-cyclopropane]-1-yl-acetic acid ethyl ester

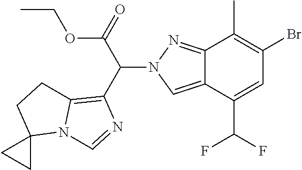 [SMILES: CCOC(=O)C(c1ncn2c1CCC21CC1)n1cc2c(C(F)F)cc(Br)c(C)c2n1]

In analogy to Example 1, step 5, (5S)-5-[2-[6-bromo-4-(difluoromethyl)-7-methyl-indazol-2-yl]-3-ethoxy-3-keto-propanoyl]-4-azaspiro[2.4]heptane-4-carboxylic acid tert-butyl ester was deprotected using HCl in dioxane followed by reaction with potassium thiocyanate to give the crude intermediate which was used for the next step without further purification.

In analogy to Example 7, step 6, the intermediate was treated with hydrogen peroxide in AcOH to give the title compound as a light yellow foam. MS: m/e=481.2 ([M+H]⁺)

### Step 3:2-[6-Bromo-4-(difluoromethyl)-7-methyl-indazol-2-yl]-2-spiro[6,7-dihydropyrrol[1,2-c]imidazole-5,1′-cyclopropane]-1-yl-N-thiazol-2-yl-acetamide

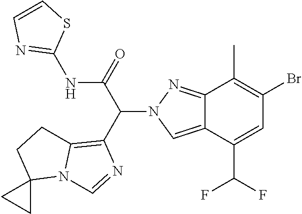 [SMILES: Cc1c(Br)cc(C(F)F)c2cn(C(C(=O)Nc3nccs3)c3ncn4c3CCC43CC3)nc12]

The title compound was obtained as a light yellow foam, MS: m/e=535.0 (M+H⁺), using chemistry similar to that described in Example 7, step 7 starting from 2-[6-bromo-4-(difluoromethyl)-7-methyl-indazol-2-yl]-2-spiro[6,7-dihydropyrrol[1,2-c]imidazole-5,1′-cyclopropane]-1-yl-acetic acid ethyl ester (Example 6, step 2) and thiazol-2-amine.

### Step 4:2-[4-(Difluoromethyl)-6-[2-(4-formylphenyl)ethynyl]-7-methyl-indazol-2-yl]-2-spiro[6,7-dihydropyrrol[1,2-c]imidazole-5,1′-cyclopropane]-1-yl-N-thiazol-2-yl-acetamide

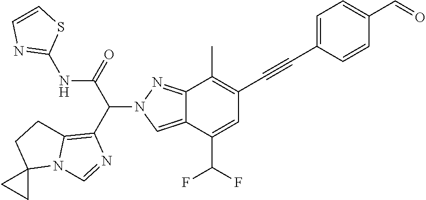 [SMILES: Cc1c(C#Cc2ccc(C=O)cc2)cc(C(F)F)c2cn(C(C(=O)Nc3nccs3)c3ncn4c3CCC43CC3)nc12]

The title compound was obtained as a dark brown amorphous, MS: m/e=583.3 (M+H⁺), using chemistry similar to that described in Example 7, step 10 starting from 2-[6-bromo-4-(difluoromethyl)-7-methyl-indazol-2-yl]-2-spiro[6,7-dihydropyrrol[1,2-c]imidazole-5,1′-cyclopropane]-1-yl-N-thiazol-2-yl-acetamide (Example 6, step 3) and 4-ethynylbenzaldehyde.

### Step 5:2-[4-(Difluoromethyl)-6-[2-[4-[[4-(hydroxymethyl)-1-piperidyl]methyl]phenyl]ethynyl]-7-methyl-indazol-2-yl]-2-(5-ethyl-5-methyl-6,7-dihydropyrrolo[1,2-c]imidazol-1-yl)-N-thiazol-2-yl-acetamide

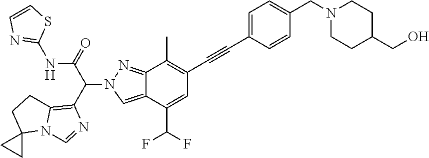 [SMILES: Cc1c(C#Cc2ccc(CN3CCC(CO)CC3)cc2)cc(C(F)F)c2cn(C(C(=O)Nc3nccs3)c3ncn4c3CCC43CC3)nc12]

The title compound was obtained as a light yellow solid, MS: m/e=682.5 (M+H⁺), using chemistry similar to that described in Example 7, step 8 starting from 2-[4-(difluoromethyl)-6-[2-(4-formylphenyl)ethynyl]-7-methyl-indazol-2-yl]-2-spiro[6,7-dihydropyrrol[1,2-c]imidazole-5,1′-cyclopropane]-1-yl-N-thiazol-2-yl-acetamide (Example 6, step 4) and piperidin-4-ylmethanol.

### Example 7

### 2-[4-(Difluoromethyl)-6-[2-[4-[[4-(hydroxymethyl)-1-piperidyl]methyl]phenyl]ethynyl]-7-methyl-indazol-2-yl]-2-[(6R)-6-fluoro-6,7-dihydro-5H-pyrrolo[1,2-c]imidazol-1-yl]-N-thiazol-2-yl-acetamide

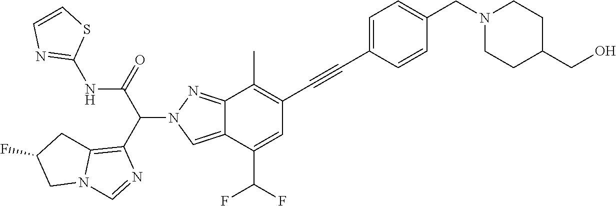 [SMILES: Cc1c(C#Cc2ccc(CN3CCC(CO)CC3)cc2)cc(C(F)F)c2cn(C(C(=O)Nc3nccs3)c3ncn4c3C[C@@H](F)C4)nc12]

### Step 1:1-Bromo-5-(difluoromethyl)-3-fluoro-2-methylbenzene

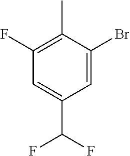

A solution of 3-bromo-5-fluoro-4-methylbenzaldehyde (CAS 1370411-47-4, 20.5 g, 89.7 mmol, Eq: 1.0) in DCM (98 mL) was cooled with ice bath. Morpholinosulfur trifluoride (CAS 51010-74-3, 24.8 g, 17.3 mL, 135 mmol, Eq: 1.5) was added in portions. The reaction mixture was stirred at 0-5° C. for 20 min, then stirred for 16 hours at room temperature. With ice cooling, saturated aqueous NaHCO₃ (300 mL) was added carefully. The reaction mixture was stirred for 1 hour at room temperature. The reaction mixture was poured into DCM and washed with water. The organic layer was dried over Na₂SO₄ and concentrated in vacuo. The crude material was purified by flash chromatography (silica gel, 120 g, 100% pentane) to give the title compound as a colourless oil (18.6 g, 87% yield). ¹H NMR (300 MHz, chloroform-d) δ=7.50 (s, 1H), 7.16 (d, J=9.1 Hz, 1H), 6.57 (t, J=56.0 Hz, 1H), 2.50-2.22 (m, 3H)

### Step 2:6-Bromo-4-(difluoromethyl)-7-methyl-1H-indazole

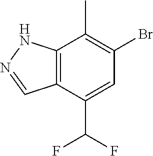

A solution of 1-bromo-5-(difluoromethyl)-3-fluoro-2-methylbenzene (Example 1, step 1) (26.4 g, 110 mmol, Eq: 1.0) in THF (240 mL) was cooled to −75° C. LDA (2 M in THF/heptane/ethylbenzene, 66.3 mL, 133 mmol, Eq: 1.2) was added dropwise below −70° C. The reaction mixture was stirred for 30 min at −75° C. Ethyl formate (16.4 g, 17.7 mL, 220 mmol, Eq: 2.0) was added below −70° C. The reaction mixture was stirred at −75° C. for 30 min. AcOH (16.6 g, 15.8 mL, 277 mmol, Eq: 2.5) was added below −55° C. The reaction mixture was allowed to warm up to room temperature and poured into EtOAc and washed with dilute aqueous HCl, water and brine. The organic layer was dried over Na₂SO₄ and concentrated in vacuo to give presumed 4-bromo-6-(difluoromethyl)-2-fluoro-3-methylbenzaldehyde as a yellow oil (29.5 g) which was used without further purification.

The crude presumed 4-bromo-6-(difluoromethyl)-2-fluoro-3-methylbenzaldehyde (29.5 g) was dissolved in DME (150 mL). O-Methylhydroxylamine hydrochloride (10.2 g, 122 mmol, Eq: 1.84) and K₂CO₃ (30.6 g, 221 mmol, Eq: 3.34) were added. The reaction mixture was stirred for 2.5 hours at 45° C. then filtered through sintered glass and washed with DME (2×). The filtrate was concentrated in vacuo. The oxime ether intermediate was dissolved in DMSO (150 mL). Hydrazine hydrate (83 g, 80.5 mL, 1.66 mol, Eq: 25) was added. The reaction mixture was stirred for 3 hours at 110° C. The reaction mixture was poured into EtOAc/THF 5:1 and washed with water and brine. The organic layer was dried over Na₂SO₄ and concentrated in vacuo. The crude material was purified by flash chromatography (silica gel, 2×120 g, 0% to 30% EtOAc in heptane) to give the title compound as a white solid (13.5 g, 74% yield). m/z 258.9, 260.8 [M+H]⁺, ESI pos, Br isotopes.

### Step 3: Ethyl 2-[6-bromo-4-(difluoromethyl)-7-methyl-indazol-2-yl]acetate

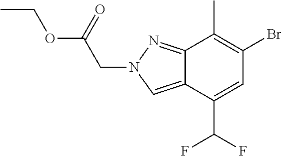 [SMILES: CCOC(=O)Cn1cc2c(C(F)F)cc(Br)c(C)c2n1]

To a solution of 6-bromo-4-(difluoromethyl)-7-methyl-1H-indazole (Example 1, step 2) (19 g, 72.8 mmol, Eq: 1.0) in DMF (75 mL) was added ethyl 2-bromoacetate (18.2 g, 12.2 mL, 109 mmol, Eq: 1.5). The reaction mixture was stirred for 16 hours at 100° C. The reaction mixture was poured into EtOAc and washed with water and brine. The organic layer was dried over Na₂SO₄ and concentrated in vacuo. The crude material was purified by flash chromatography (silica gel, 2 ×120 g, 0% to 20% EtOAc in heptane) to give the title compound as a yellow solid (21.2 g, 80% yield). m/z 346.9, 348.8, [M+H]⁺, ESI pos, Br isotopes.

### Step 4: tert-Butyl (2S,4R)-2-[2-[6-bromo-4-(difluoromethyl)-7-methyl-indazol-2-yl]-3-ethoxy-3-oxo-propanoyl]-4-fluoro-pyrrolidine-1-carboxylate

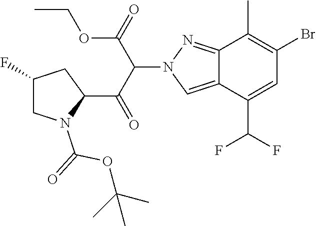 [SMILES: CCOC(=O)C(C(=O)[C@@H]1C[C@@H](F)CN1C(=O)OC(C)(C)C)n1cc2c(C(F)F)cc(Br)c(C)c2n1]

### Preparation of tert-butyl (2S,4R)-4-fluoro-2-(imidazole-1-carbonyl) pyrrolidine-1-carboxylate

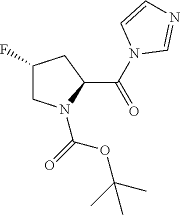 [SMILES: CC(C)(C)OC(=O)N1C[C@H](F)C[C@H]1C(=O)n1ccnc1]

To a solution of (2S,4R)-1-tert-butoxycarbonyl-4-fluoro-pyrrolidine-2-carboxylic acid (CAS 203866-14-2, 30 g, 129 mmol, Eq: 1.0) in DCM (300 mL) was added 1,1′-carbonyldiimidazole (25 g, 154 mmol, Eq: 1.2) in portions at 0° C. The reaction mixture was stirred for 3 hours at room temperature. The reaction mixture was washed with water (3×) and 1M aqueous NaHCO₃ (1×). The organic layer was dried over Na₂SO₄ and concentrated in vacuo at 30° C. to give tert-butyl (2S,4R)-4-fluoro-2-(imidazole-1-carbonyl) pyrrolidine-1-carboxylate (36.6 g, 129 mmol, 100% yield) as a white solid which was stored at −20° C. prior to use. ¹H NMR (chloroform-d, 300 MHz) δ 8.27 (s, 1H), 7.56 (br d, 1H, J=1.4 Hz), 7.15 (br d, 1H, J=12.1 Hz), 4.9-5.2 (m, 1H), 3.6-4.1 (m, 2H), 2.0-2.9 (m, 2H), 1.2-1.5 (m, 9H).

KO^{t}Bu (4.53 g, 40.3 mmol, Eq: 2.0) was dissolved in THF (18 mL). The reaction mixture was cooled to −55° C. A solution of ethyl 2-[6-bromo-4-(difluoromethyl)-7-methyl-indazol-2-yl]acetate (Example 1, step 3) (7 g, 20.2 mmol, Eq: 1.0) in THF (24 mL) was added dropwise below −50° C. The reaction mixture was stirred for 1 hour between −50° C. and −55° C. A solution of previously-prepared tert-butyl (2S,4R)-4-fluoro-2-(imidazole-1-carbonyl) pyrrolidine-1-carboxylate (6.85 g, 24.2 mmol, Eq: 1.2) in THF (50 mL) was added dropwise below −50° C. The reaction mixture was stirred for 15 min at −50° C., then allowed to warm up to −30° C. 10% aqueous citric acid (60 mL) was added below −20° C., and the mixture was stirred for 1 hour at 0

C. The reaction mixture was poured into EtOAc and washed with water and brine. The organic layer was dried over Na₂SO₄ and concentrated in vacuo at 30° C. to give the title compound as a yellow amorphous semisolid (12.9 g, 20.2 mmol, 88% purity, 100% yield). m/z 562.1, 563.9 [M+H]⁺, ESI pos, Br isotopes.

### Step 5: Ethyl 2-[6-bromo-4-(difluoromethyl)-7-methyl-indazol-2-yl]-2-[(6R)-6-fluoro-3-thioxo-2,5,6,7-tetrahydropyrrolo[1,2-c]imidazol-1-yl]acetate

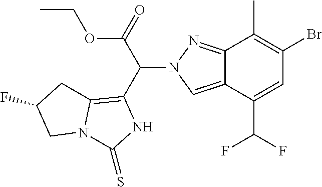 [SMILES: CCOC(=O)C(c1[nH]c(=S)n2c1C[C@@H](F)C2)n1cc2c(C(F)F)cc(Br)c(C)c2n1]

To a solution of tert-butyl (2S,4R)-2-[2-[6-bromo-4-(difluoromethyl)-7-methyl-indazol-2-yl]-3-ethoxy-3-oxo-propanoyl]-4-fluoro-pyrrolidine-1-carboxylate (Example 1, step 4) (12.9 g, 20.2 mmol, Eq: 1.0) in ethanol (24 mL) was added HCl (1.25 M in ethanol, 80.6 mL, 101 mmol, Eq: 5.0). The reaction mixture was stirred for 1 hour at 55° C. The reaction mixture was cooled to rt, then water (6 mL) and potassium thiocyanate (2.55 g, 26.2 mmol, Eq: 1.3) were added. The reaction mixture was stirred for 30 min at rt. The ethanol was removed in vacuo at 30° C., and pyridine (23.9 g, 24.5 mL, 302 mmol, Eq: 15) was added. The reaction mixture was stirred at room temperature for 75 min. The reaction mixture was poured into EtOAc and washed with 2 N aqueous HCl (until the aqueous phase was pH 1), water and brine. The organic layer was dried over Na₂SO₄ and concentrated in vacuo to give the title compound as a yellow semisolid (9.65 g, 60% purity, 57% yield). m/z 502.9, 505.9 [M+H]⁺, ESI pos, Br isotopes.

### Step 6: Ethyl 2-[6-bromo-4-(difluoromethyl)-7-methyl-indazol-2-yl]-2-[(6R)-6-fluoro-6,7-dihydro-5H-pyrrolo[1,2-c]imidazol-1-yl]acetate

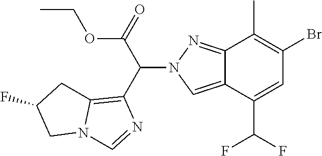 [SMILES: CCOC(=O)C(c1ncn2c1C[C@@H](F)C2)n1cc2c(C(F)F)cc(Br)c(C)c2n1]

To a suspension of p-toluenesulfonic acid monohydrate (10.9 g, 57.5 mmol, Eq: 5.0) in acetonitrile (70 mL) was added hydrogen peroxide (35% aq., 8.38 g, 7.42 mL, 86.3 mmol, Eq: 7.5) dropwise at 0-3° C. to give a colorless solution. After 10 min, ethyl 2-[6-bromo-4-(difluoromethyl)-7-methyl-indazol-2-yl]-2-[(6R)-6-fluoro-3-thioxo-2,5,6,7-tetrahydropyrrolo[1,2-c]imidazol-1-yl]acetate (Example 1, step 5) (9.65 g, 11.5 mmol, Eq: 1.0) in acetonitrile (28 mL) was added dropwise below 8° C. The reaction mixture was stirred for 1.5 hours at 0° C. The reaction mixture was poured into EtOAc and washed with Na₂CO₃ solution and brine. The organic layer was dried over Na₂SO₄ and concentrated in vacuo. The crude material was purified by flash chromatography (silica gel, 120 g, 0% to 60% (EtOAc/EtOH/aq. NH₃ 75:25:2) in heptane) to give the title compound as a yellow foam (3.96 g, 73% yield). m/z 469.1, 471.1 [M+H]⁺, ESI pos.

### Step 7:2-[6-Bromo-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide

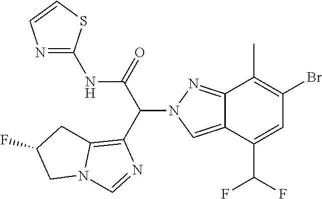 [SMILES: Cc1c(Br)cc(C(F)F)c2cn(C(C(=O)Nc3nccs3)c3ncn4c3C[C@@H](F)C4)nc12]

Ethyl 2-[6-bromo-4-(difluoromethyl)-7-methyl-indazol-2-yl]-2-[(6R)-6-fluoro-6,7-dihydro-5H-pyrrolo[1,2-c]imidazol-1-yl]acetate (Example 1, step 6) (200 mg, 0.424 mmol) was dissolved in 2 ml of methanol and 2 ml of THF. LiOH (1M in water) (0.4 ml, 0.424 mmol, Eq: 1.0) was added at room temperature. The mixture was stirred for 2 hours at room temperature. The reaction mixture was concentrated in vacuo to dryness and the residue was dissolved in 2 ml of DMF. Thiazol-2-amine (42 mg, 0.424 mmol, Eq: 1.0), Hunig's base (0.37 ml, 2.12 mmol, Eq: 5.0) and HATU (194 mg, 0.509 mmol, Eq: 1.2) were added at room temperature. The mixture was stirred at room temperature for 90 minutes. The reaction mixture was extracted with water and two times with ethyl acetate. The organic layers were extracted with water, dried over sodium sulfate and concentrated to dryness. The crude product was purified by flash chromatography on a silica gel column eluting with a dichloromethane:methanol 100:0 to 90:10 gradient to obtain the desired product (115 mg, 49% yield) as a light yellow solid, MS: m/e=527.1 (M+H⁺).

### Step 8: [1-[(4-Ethynylphenyl)methyl]-4-piperidyl]methanol

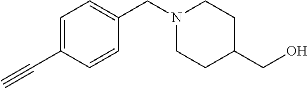 [SMILES: C#Cc1ccc(CN2CCC(CO)CC2)cc1]

4-Ethynylbenzaldehyde (20.5 g, 157.5 mmol) was dissolved in 525 ml of dichloromethane. Piperidin-4-ylmethanol (20 g, 173.2 mmol, Eq: 1.1) and sodium triacetoxyborohydride (53.4 g, 252.0 mmol, Eq: 1.6) were added at room temperature. The mixture was stirred at room temperature for 4 hours. The reaction mixture was extracted with 1M sodium carbonate solution and two times with dichloromethane. The organic layers were dried over sodium sulfate and concentrated to dryness to obtain the desired product (34.8 g, 91% yield) as a light yellow solid, MS: m/e=527.1 (M+H⁺).

### Step 9: [1-[(4-Ethynylphenyl)methyl]-4-piperidyl]methanol hydrochloride

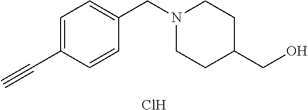 [SMILES: C#Cc1ccc(CN2CCC(CO)CC2)cc1 ; Cl]

[1-[(4-Ethynylphenyl)methyl]-4-piperidyl]methanol (Example 1, step 8) (34.8 g) was dissolved in 200 ml of tetrahydrofuran. 4 M Hydrogen chloride solution in 1,4-dioxane (39.4 ml, 158 mmol, Eq: 1.0) was added drop wise at 10-20° C. A white precipitate was formed and stirred for 2 hours. The precipitate was collected by filtration, washed with three 50 ml portions of tetrahydrofuran and dried in vacuo to give the title compound (38.6 g, 92%) as a white solid, MS: m/e=527.1 (M+H⁺).

### Step 10:2-[4-(Difluoromethyl)-6-[2-[4-[[4-(hydroxymethyl)-1-piperidyl]methyl]phenyl]ethynyl]-7-methyl-indazol-2-yl]-2-[(6R)-6-fluoro-6,7-dihydro-5H-pyrrolo[1,2-c]imidazol-1-yl]-N-thiazol-2-yl-acetamide

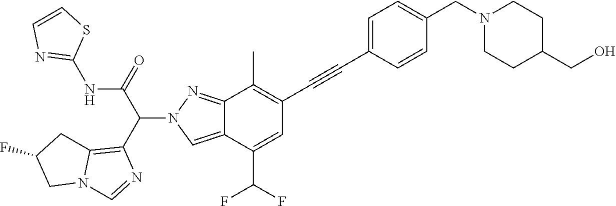 [SMILES: Cc1c(C#Cc2ccc(CN3CCC(CO)CC3)cc2)cc(C(F)F)c2cn(C(C(=O)Nc3nccs3)c3ncn4c3C[C@@H](F)C4)nc12]

2-[6-Bromo-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide (Example 1, step 7) (100 mg, 0.19 mmol) and [1-[(4-ethynylphenyl)methyl]-4-piperidyl]methanol hydrochloride (Example 1, step 9) (76 mg, 0.286 mmol, Eq: 1.5) were dissolved in 5 ml of DMF. Triethylamine (0.1 ml, 0.76 mmol, Eq: 4.0), bis-(triphenylphosphine)-palladium (II) dichloride (7 mg, 0.01 mmol, Eq: 0.05), triphenylphosphine (5 mg, 0.019 mmol, Eq: 0.1) and copper (I) iodide (2 mg, 0.01 mmol, Eq: 0.05) were added and the mixture was stirred for 2 hours at 80° C. The reaction mixture was extracted with water and three times with dichloromethane. The organic layers were dried over sodium sulfate and concentrated to dryness. The crude product was purified by flash chromatography on a silica gel column eluting with a dichloromethane:methanol 100:0 to 75:25 gradient to obtain the desired product (7 mg, 5% yield) as a light yellow oil, MS: m/e=674.5 (M+H⁺).

### Example 8—In Vitro Characterization

### Cell Lines

Ba/F3 cell lines stably expressing the EGFR mutants L858R (#2039), L858R/C797S (#C2052) and L858R/T790M/C797S (#2056) were purchased from CrownBio. NCI-H1975 cells (#CRL-5908), NCI-H2073 cells (#CRL-5918) and A431 cells (#CRL-1555) were purchased from the ATCC. NCI-H3255 cells were obtained from the NCI (#CVCL_6831). Cells were maintained in a humidified incubator at 37° C. and 5% CO₂. Ba/F3 EGFR-LR, Ba/F3 EGFR-LRTM, Ba/F3 EGFR-LRCS and Ba/F3 EGFR-LRTMCS cells were grown in RPMI 1640 GlutaMAX medium (Thermo Fisher Scientific #61870010) supplemented with 10% fetal bovine serum (FBS; VWR #07068-085). NCI-H1975, NCI-H3255, NCI-H2073, A431 and PC-9 cells were cultured in RPMI 1640 medium with ATCC modification (Thermo Fisher Scientific #A10491) supplemented with 10% FBS. Verification of cell line identities and absence of cross-contaminations by other cell lines was performed through Short Tandem Repeats-PCR analysis (only human cell lines) and MALDI-TOF analysis. Absence of mycoplasma contamination was verified through testing of antibiotic-free cultured cells for 10-14 days through the kit MycoAlert™ Mycoplasma Detection Kit (Lonza #LT07-318). All cell lines were used for no more than 20 passages after thawing for all described experiments.

### Homogeneous Time Resolved Fluorescence (HTRF) Cellular Assay

For the Homogeneous Time Resolved Fluorescence (HTRF) assays, cells were transferred into RPMI 1640 no phenol red medium (Thermo Fisher Scientific #11835063) containing 10% FBS and were plated into 384-well plates (Greiner #781080) at a density of 15′000 cells/well (Ba/F3), 16′000 cells/well (H1975) and 10′000 cells/well (H3255) in a volume of 12 μl/well. 5′000 cells/well (H2073) and 10′000 cells/well (A-431) were plated in a volume of 11 μl/well. Control wells with medium only were also prepared. Plates were centrifuged at 300 g for 30 sec and incubated overnight at 37° C. The next day, compounds were added to concentrations ranging from 0.316 nM to 10 mM with a ¼ log dilution and DMSO (Sigma #D2650) was compensated accordingly to a final content of 0.1%. Plates were incubated at 37° C. for 4 hours. Particularly for H2073 and A-431 cells, 1 μl of 300 ng/ml EGF (final concentration 25 ng/ml) was added to each well after the 4-hour incubation and cells were incubated at 37° C. for another 5 min. The cells were eventually lysed in HTRF lysis buffer provided by the kit (see below) and stored at −80° C. until further use. HTRF assay for pEGFR (CisBio #64EG1PEH) or pERK (CisBio #64AERPEH) was then performed according to the manufacturer's instructions.

### Cell Viability Assay

For the cell viability assays, Ba/F3 cells in growth medium containing 10% FBS were plated in 384-well black clear bottom plates (Falcon #353962) at 2′000 cells/well in 50 μl, and compounds were added to concentrations ranging from 0.316 nM to 10 mM with a ¼ log dilution and DMSO (Sigma #D2650) was compensated accordingly to a final content of 0.1%. Plates were incubated for 72 hours at 37° C. After treatment with compounds, 25 μl/well CellTiter-Glo 2.0 reagent (Promega #G9243) were added and plates were incubated for 10 min at RT and luminescence was quantified according to the manufacturer's protocol.

### In Vitro Metabolic Stability

The in vitro metabolic stability was assessed in human and rat short-term hepatocytes suspensions. Primary pooled cryopreserved hepatocytes obtained from BioIVT (Westbury, NY, USA) were thawed and suspended in William's E medium supplemented with 10% fetal bovine serum. The suspended hepatocytes were pre-incubated at 37° C. (5% CO2) and reactions were initiated by adding the compound. Final assay conditions were 1 μM compound (final assay solvent concentration 0.01% DMSO), 1×10⁶ hepatocytes/mL incubate. The mixtures were then incubated up to 2 hours at 37° C.′ (5% CO2). The reactions were stopped by placing samples on dry ice before storage at −80° C. until analysis by LC-MS/MS. The natural logarithm of the percentage initial drug concentration in the incubation samples was plotted against time and a linear regression analysis was applied using GraphPad Prism version 7.04 for Windows (GraphPad Software, La Jolla, CA) to define the slope. The in vitro hepatocyte intrinsic hepatocyte clearance rate (CLᵢₙₜ) was calculated from the slope of the linear regression,-slope, with V representing the volume of the incubation in μL and N as the number of hepatocytes per incubation as detailed in Equation X:

$$In Vitro CLint ( \frac{\frac{uL}{min}}{million} hepatocytes ) = \frac{- slope * V * million hepatocytes}{N}$$

The results of the various in vitro assays are shown in Tables 1 and 2

TABLE 1
In vitro data of Examples 1-6 and Reference
Compounds JBJ-04-125-02 and EAI045

| | pEGFR | | Viability | | | |
|---|---|---|---|---|---|---|
| | IC₅₀ (nM) | | IC₅₀/IC₉₀ (nM) | | | |
| | L858R/ | | L858R/ | | CLᵢₙₜ(μL/min/10⁶ | |
| | T790M/ | L858R/ | T790M/ | L858R/ | cells)^{c} | |
| Compound | C797S | C797S | C797S | C797S | Mouse | Human |
| Example 1 | 6 | 17 | 13/33 | 12/32 | 16 | 3.5 |
| Example 2 | 7 | 8 | 8/19 | 4/10 | 21 | 6.1 |
| Example 3 | 10 | 20 | 16/38 | 3/8 | 25 | 5.9 |
| Example 4 | 6 | 106 | 9/22 | 800/2′800 | n.a. | n.a. |
| Example 5 | 3 | 23 | 1/3 | 2/5 | 36 | 2.4 |
| Example 6 | 2 | 20 | 1/2 | 2/2 | 25 | 1 |
| JBJ-04-125-02 | 10 | 100 | 30/40 | 1′550/7′280 | n.a. | n.a. |
| EAI045 | 6 | 460 | 424/2251 | >3′300/>3′300 | n.a. | n.a. |

^{a} IC₅₀ for pEGFR inhibition determined in Ba/F3 cell lines stably expressing EGFR^{L858R/C797S} and EGFR^{L858R/T790M/C797S}, respectively, with HTRF assay.
^{b} IC₉₀ and IC₅₀ determined in Ba/F3 cell lines stably expressing EGFR^{L858R/C797S} and EGFR^{L858R/T790M/C797S}, respectively, via cell titer glo assay.
^{c}Intrinsic clearance in liver hepatocytes.

Reference compounds EAI045 (left) and JBJ-04-125-02 (right):

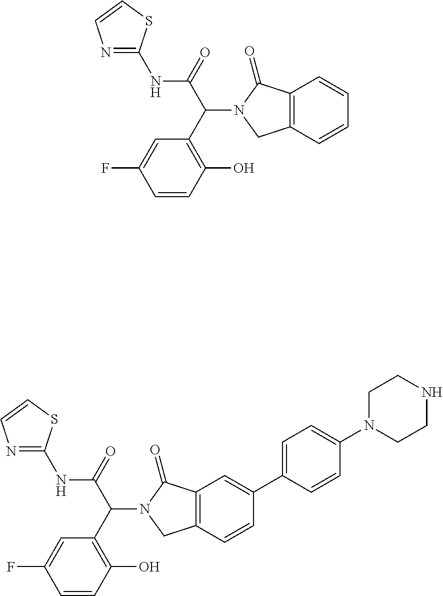 [SMILES: O=C(Nc1nccs1)C(c1cc(F)ccc1O)N1Cc2ccccc2C1=O ; O=C(Nc1nccs1)C(c1cc(F)ccc1O)N1Cc2ccc(-c3ccc(N4CCNCC4)cc3)cc2C1=O]

TABLE 2
IC₅₀ (nM) values of EGFR inhibitors in human cancer cells
expressing WT EGFR, EGFR^{L858R} or EGFR^{L858R/T790M}.

| | H2073 | A431 | H3255 | H1975 |
|---|---|---|---|---|
| Compound | EGFR WT | EGFR WT | EGFR LR | EGFR LRTM |
| Example 1 | ~2′000 | ~2.500 | 16 | 7 |
| Example 6 | 545 | 577 | 16 | 8 |
| JBJ-04-125-02 | 3′910 | 4′213 | 428 | 11 |
| Osimertinib | 240 | 322 | 40 | 30 |
| Erlotinib | 17* | 92* | 40 | >5′000 |

Orthosteric EGFR inhibitors Osimertinib (left) and Erlotinib (right):

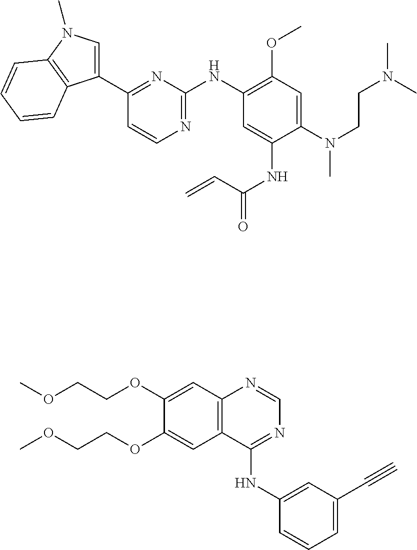

### Example 9—In Vivo Efficacy Studies

The studies were conducted at Charles River Discovery Research Service GmbH, Germany.

Mice were maintained under pathogen-free conditions with daily cycles of 12-hour light/12-hour darkness. Experiments were performed in accordance with animal welfare guidelines (Federation of European Laboratory Animal Science Associations), and study protocols were reviewed and approved by the local government.

Subcutaneous cell line derived models with Ba/F3 cell clones were established through cell injection (1×10⁷) to the right flank of female Balb/c nude mice. Compound treatment was initiated after randomization when tumors reached 100-200 mm³ in size. For the subcutaneous cell line derived xenograft model of human NSCLC NCI-H1975, 5×10⁶ cells were injected to the right flank of female Balb/c mice, which were randomized and assigned to treatment groups after tumors reached about 150 mm³. The inoculation procedure was performed with the support of a stereotactic device and anesthesia for the surgery carried out with isoflurane/O₂ using an inhalation mask.

Allosteric EGFR inhibitor Example 6 was formulated with 10% PEG400 and 5% solutol in water and osimertinib with 1% DMSO and 30% PEG300 in water. Daily oral treatments started after randomisation of mice. Individual tumor volumes were monitored through regular caliper measurement and calculated according to the formula (TV=(length×width²). Tumor volumes for respective study groups are shown as mean with SEM in mm³ along the study observation period. All raw data were exported from a local database into a tab limited csv file and processed using statistical software R and visualized with Shiny (Script written by Dr. S. Wilson, PS BIOMICS, RICB).

Data regarding primary tumor growth were statistically analyzed on study D38 (last day of control) using non-parametric methods, since the data showed asymmetrical behavior. Briefly, in a randomized two-sample design the treatment-to-control ratio (TCR) and its two-sided parametric (Fieller, 1954) or non-parametric (Hothorn and Munzel, 2000) confidence interval (CI) (1-a) were calculated. A TCR below 1.0 indicated tumor growth inhibition, with CI reflecting the data distribution. An upper CI below 1.0 confirms statistical significance. Tumor growth inhibition was calculated according to the formula: 100−[average (TVₜᵣₑₐₜₘₑₙₜ−TV_{baseline})/average (TV_{vehicle}−TV_{baseline})] based on medians. Tumor regression was calculated according to the formula: average [(TV_{baseline}−TVₜᵣₑₐₜₘₑₙₜ)/TV_{baseline})×100 based on medians. Positive values indicate tumor regression.

### Specific Numbered Embodiments

1. A combination of an allosteric EGFR inhibitor and an orthosteric EGFR inhibitor, wherein the allosteric EGFR inhibitor is a compound of formula (I)

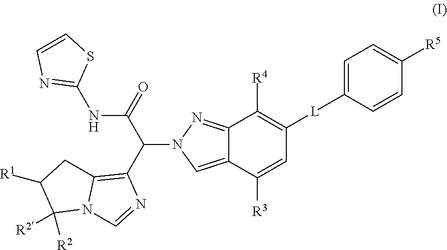

wherein
L is a bond or alkynylene;
R¹ is hydrogen or halogen;
R² and R^{2′} are independently selected from hydrogen and alkyl;
or R² and R^{2′}, together with the carbon atom to which they are attached, form cycloalkyl;
R³ is hydrogen, halogen or haloalkyl;
R⁴ is alkyl or halogen;
R⁵ is (heterocycloalkyl)alkylene or heterocycloalkyl, wherein (heterocycloalkyl)alkylene is optionally substituted with one or two substituents independently selected from R⁶, and wherein heterocycloalkyl is optionally substituted with one or two substituents independently selected from R⁷;
R⁶ is at each instance independently selected from alkyl, cycloalkyl, hydroxy and hydroxyalkyl; and
R⁷ is at each instance independently selected from alkyl, cycloalkyl, hydroxy and hydroxyalkyl;
or a pharmaceutically acceptable salt thereof.
2. A combination according to embodiment 1, wherein the allosteric EGFR inhibitor is a compound of formula (I)

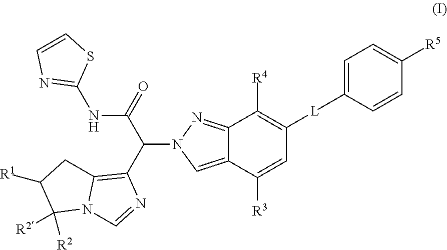

wherein
L is a bond or alkynylene;
R¹ is hydrogen or halogen;
R² and R^{2′} are independently selected from hydrogen and alkyl;
or R² and R^{2′}, together with the carbon atom to which they are attached, form cycloalkyl;
R³ is hydrogen, halogen or haloalkyl;
R⁴ is alkyl or halogen;
R⁵ is (heterocycloalkyl)alkylene or heterocycloalkyl, wherein (heterocycloalkyl)alkylene is optionally substituted with one or two substituents independently selected from R⁶, and wherein heterocycloalkyl is optionally substituted with one or two substituents independently selected from R⁷;
R⁶ is at each instance independently selected from alkyl, cycloalkyl, hydroxy and hydroxyalkyl; and
R⁷ is at each instance independently selected from alkyl, cycloalkyl, hydroxy and hydroxyalkyl.
3. A combination according to embodiment 1 or 2, wherein L is a bond or ethynylene.
4. A combination according to any one of embodiments 1 to 3, wherein R¹ is hydrogen or fluoro.
5. A combination according to any one of embodiments 1 to 4, wherein R² and R^{2′} are hydrogen or together with the carbon atom to which they are attached, form cyclopropyl.
6. A combination according to any one of embodiments 1 to 5, wherein R³ is halogen or haloalkyl.
7. A combination according to any one of embodiments 1 to 6, wherein R³ is chloro or difluoromethyl.
8. A combination according to any one of embodiments 1 to 7, wherein R⁴ is methyl or chloro.
9. A combination according to any one of embodiments 1 to 8, wherein R⁵ is morpholinyl or piperidinylmethylene optionally substituted with hydroxymethyl.
10. A combination according to any one of embodiments 1 to 9, wherein the compound of formula (I) is selected from
   2-[4,7-dichloro-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
   2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
   2-[4-(difluoromethyl)-7-methyl-6-(4-morpholinophenyl)indazol-2-yl]-2-spiro[6,7-dihydropyrrol[1,2-c]imidazole-5,1′-cyclopropane]-1-yl-N-thiazol-2-yl-acetamide;
   2-[4-(difluoromethyl)-6-[2-[4-[[4-(hydroxymethyl)-1-piperidyl]methyl]phenyl]ethynyl]-7-methyl-indazol-2-yl]-2-(6,7-dihydro-5H-pyrrolo[1,2-c]imidazol-1-yl)-N-thiazol-2-yl-acetamide; and
   2-[4-(difluoromethyl)-6-[2-[4-[[4-(hydroxymethyl)-1-piperidyl]methyl]phenyl]ethynyl]-7-methyl-indazol-2-yl]-2-(5-ethyl-5-methyl-6,7-dihydropyrrolo[1,2-c]imidazol-1-yl)-N-thiazol-2-yl-acetamide.
11. A combination according to any one of embodiments 1 to 9, wherein the compound of formula (I) is 2-[4,7-dichloro-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide.
12. A combination according to any one of embodiments 1 to 9, wherein the compound of formula (I) is 2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide.
13. A combination according to any one of embodiments 1 to 9, wherein the compound of formula (I) is 2-[4-(difluoromethyl)-7-methyl-6-(4-morpholinophenyl)indazol-2-yl]-2-spiro[6,7-dihydropyrrol[1,2-c]imidazole-5,1′-cyclopropane]-1-yl-N-thiazol-2-yl-acetamide.
14. A combination according to any one of embodiments 1 to 9, wherein the compound of formula (I) is 2-[4-(difluoromethyl)-6-[2-[4-[[4-(hydroxymethyl)-1-piperidyl]methyl]phenyl]ethynyl]-7-methyl-indazol-2-yl]-2-(6,7-dihydro-5H-pyrrolo[1,2-c]imidazol-1-yl)-N-thiazol-2-yl-acetamide.
15. A combination according to any one of embodiments 1 to 9, wherein the compound of formula (I) is 2-[4-(difluoromethyl)-6-[2-[4-[[4-(hydroxymethyl)-1-piperidyl]methyl]phenyl]ethynyl]-7-methyl-indazol-2-yl]-2-(5-ethyl-5-methyl-6,7-dihydropyrrolo[1,2-c]imidazol-1-yl)-N-thiazol-2-yl-acetamide.
16. A combination according to any one of embodiments 1 to 15, wherein the orthosteric EGFR inhibitor is selected from cetuximab, panitumumab, osimertinib, erlotinib, gefitinib, necitumumab, neratinib, lapatinib, vandetanib, brigatinib, almonertinib, lazertinib and furmonertinib, or a pharmaceutically acceptable salt thereof, in particular selected from osimertinib, almonertinib, lazertinib and furmonertinib, or a pharmaceutically acceptable salt thereof.
17. A combination according to any one of embodiments 1 to 16, wherein the orthosteric EGFR inhibitor is osimertinib, or a pharmaceutically acceptable salt thereof.
18. A combination according to any one of embodiments 1 to 16, wherein the orthosteric EGFR inhibitor is almonertinib, or a pharmaceutically acceptable salt thereof.
19. A combination according to any one of embodiments 1 to 16, wherein the orthosteric EGFR inhibitor is lazertininb, or a pharmaceutically acceptable salt thereof.
20. A combination according to any one of embodiments 1 to 16, wherein the orthosteric EGFR inhibitor is furmonertinib, or a pharmaceutically acceptable salt thereof.
21. A combination according to any one of embodiments 1 to 16, for use as therapeutically active substance.
22. A pharmaceutical composition comprising a combination according to any one of embodiments 1 to 20 and a therapeutically inert carrier.
23. A combination according to any one of embodiments 1 to 20 for use in the treatment or prophylaxis of cancer, in particular non-small cell lung cancer.
24. The use of a combination according to any one of embodiments 1 to 20 for the treatment or prophylaxis of cancer, in particular non-small cell lung cancer.
25. The use of a combination according to any one of embodiments 1 to 20 for the preparation of a medicament for the treatment or prophylaxis of cancer, in particular non-small cell lung cancer.
26. The use of a combination according to any one of embodiments 1 to 20 for the preparation of a medicament for the treatment or prophylaxis of non-small cell lung cancer.
27. A method for the treatment or prophylaxis of cancer, in particular non-small cell lung cancer, which method comprises administering an effective amount of a combination according to any one of embodiments 1 to 20 to a patient in need thereof.
28. A combination, a use, a method or a pharmaceutical composition according to any one of embodiments 21 to 27, wherein the allosteric EGFR inhibitor and the orthosteric EGFR inhibitor are both administered orally.
29. A combination, a use, a method or a pharmaceutical composition according to any one of embodiments 21 to 28, wherein the allosteric EGFR inhibitor is administered concurrently with the orthosteric EGFR inhibitor.
30. A combination, a use, a method or a pharmaceutical composition according to any one of embodiments 21 to 29, wherein the allosteric EGFR inhibitor and the orthosteric EGFR inhibitor are co-formulated.
31. A combination, a use, a method or a pharmaceutical composition according to any one of embodiments 21 to 28, wherein the allosteric EGFR inhibitor and the orthosteric EGFR inhibitor are administered sequentially.
32. A combination, a use, a method or a pharmaceutical composition according to any one of embodiments 23 to 27, wherein the cancer is associated with at least one EGFR mutation selected from del19, L858R, T790M and C797S.
33. A combination, a use, a method or a pharmaceutical composition according to any one of embodiments 23 to 27, wherein the cancer is associated with at least two EGFR mutations selected from del19, L858R, T790M and C797S.

1. A combination of an allosteric EGFR inhibitor and an orthosteric EGFR inhibitor, wherein the allosteric EGFR inhibitor is a compound of formula (I) 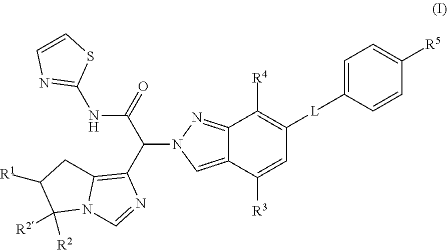
wherein
L is a bond or alkynylene;
R¹ is hydrogen or halogen;
R² and R^{2′} are independently selected from hydrogen and alkyl;
or R² and R^{2′}, together with the carbon atom to which they are attached, form cycloalkyl;
R³ is hydrogen, halogen or haloalkyl;
R⁴ is alkyl or halogen;
R⁵ is (heterocycloalkyl)alkylene or heterocycloalkyl, wherein (heterocycloalkyl)alkylene is optionally substituted with one or two substituents independently selected from R⁶, and wherein heterocycloalkyl is optionally substituted with one or two substituents independently selected from R⁷;
R⁶ is at each instance independently selected from alkyl, cycloalkyl, hydroxy and hydroxyalkyl; and
R⁷ is at each instance independently selected from alkyl, cycloalkyl, hydroxy and hydroxyalkyl;
or a pharmaceutically acceptable salt thereof.

2. The combination according to claim 1, wherein the allosteric EGFR inhibitor is a compound of formula (I): 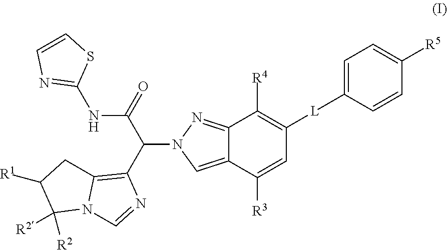
wherein
L is a bond or alkynylene;
R¹ is hydrogen or halogen;
R² and R^{2′} are independently selected from hydrogen and alkyl;
or R² and R^{2′}, together with the carbon atom to which they are attached, form cycloalkyl;
R³ is hydrogen, halogen or haloalkyl;
R⁴ is alkyl or halogen;
R⁵ is (heterocycloalkyl)alkylene or heterocycloalkyl, wherein (heterocycloalkyl)alkylene is optionally substituted with one or two substituents independently selected from R⁶, and wherein heterocycloalkyl is optionally substituted with one or two substituents independently selected from R⁷;
R⁶ is at each instance independently selected from alkyl, cycloalkyl, hydroxy and hydroxyalkyl; and
R⁷ is at each instance independently selected from alkyl, cycloalkyl, hydroxy and hydroxyalkyl.

3. The combination according to claim 1, wherein L is a bond or ethynylene.

4. The combination according to claim 1, wherein R¹ is hydrogen or fluoro.

5. The combination according to claim 1, wherein R² and R^{2′} are hydrogen or together with the carbon atom to which they are attached, form cyclopropyl.

6. (canceled)

7. The combination according to claim 1, wherein R³ is chloro or difluoromethyl.

8. The combination according to claim 1, wherein R⁴ is methyl or chloro.

9. The combination according to claim 1, wherein R⁵ is morpholinyl or piperidinylmethylene optionally substituted with hydroxymethyl.

10. The combination according to claim 1, wherein the compound of formula (I) is selected from
2-[4,7-dichloro-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-(4-morpholinophenyl)indazol-2-yl]-2-spiro[6,7-dihydropyrrol[1,2-c]imidazole-5,1′-cyclopropane]-1-yl-N-thiazol-2-yl-acetamide;
2-[4-(difluoromethyl)-6-[2-[[4-[4-(hydroxymethyl)-1-piperidyl]methyl]phenyl]ethynyl]-7-methyl-indazol-2-yl]-2-(6,7-dihydro-5H-pyrrolo[1,2-c]imidazol-1-yl)-N-thiazol-2-yl-acetamide; and
2-(4-(difluoromethyl)-6-((4-((4-(hydroxymethyl)piperidin-1-yl)methyl)phenyl)ethynyl)-7-methyl-2H-indazol-2-yl)-2-(6′,7′-dihydrospiro[cyclopropane-1,5′-pyrrolo[1,2-c]imidazol]-1′-yl)-N-(thiazol-2-yl)acetamide;
or a pharmaceutically acceptable salt thereof.

11. (canceled)

12. (canceled)

13. The combination according to claim 1, wherein the orthosteric EGFR inhibitor is selected from cetuximab, panitumumab, osimertinib, erlotinib, gefitinib, necitumumab, neratinib, lapatinib, vandetanib, brigatinib, almonertinib, lazertinib and furmonertinib, or a pharmaceutically acceptable salt thereof, in particular selected from osimertinib, almonertinib, lazertinib and furmonertinib, or a pharmaceutically acceptable salt thereof.

14. The combination according to claim 1, wherein the orthosteric EGFR inhibitor is osimertinib, or a pharmaceutically acceptable salt thereof.

15. (canceled)

16. A pharmaceutical composition comprising a combination according to claim 1 and a therapeutically inert carrier.

17-20. (canceled)

21. A method for treating a cancer, comprising administering an effective amount of a combination of an allosteric EGFR inhibitor and an orthosteric EGFR inhibitor to a patient in need thereof, wherein the allosteric EGFR inhibitor is a compound of formula (I): 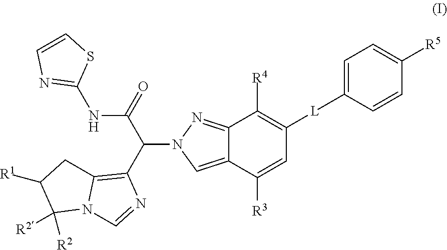
wherein
L is a bond or alkynylene;
R¹ is hydrogen or halogen;
R² and R^{2′} are independently selected from hydrogen and alkyl;
or R² and R^{2′}, together with the carbon atom to which they are attached, form cycloalkyl;
R³ is hydrogen, halogen or haloalkyl;
R⁴ is alkyl or halogen;
R⁵ is (heterocycloalkyl)alkylene or heterocycloalkyl, wherein (heterocycloalkyl)alkylene is optionally substituted with one or two substituents independently selected from R⁶, and wherein heterocycloalkyl is optionally substituted with one or two substituents independently selected from R⁷;
R⁶ is at each instance independently selected from alkyl, cycloalkyl, hydroxy and hydroxyalkyl; and
R⁷ is at each instance independently selected from alkyl, cycloalkyl, hydroxy and hydroxyalkyl;
or a pharmaceutically acceptable salt thereof.

22. The method according to claim 21, wherein the allosteric EGFR inhibitor and the orthosteric EGFR inhibitor are both administered orally.

23. The method according to claim 21, wherein the allosteric EGFR inhibitor and orthosteric EGFR inhibitor are administered concurrently or sequentially.

24. (canceled)

25. (canceled)

26. The method according to claim 21, wherein the cancer is associated with at least one EGFR mutation selected from del19, L858R, T790M and C797S.

27. (canceled)

28. (canceled)

29. The method according to claim 21, wherein the allosteric EGFR inhibitor is selected from:
2-[4,7-dichloro-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-(4-morpholinophenyl)indazol-2-yl]-2-spiro[6,7-dihydropyrrol[1,2-c]imidazole-5,1′-cyclopropane]-1-yl-N-thiazol-2-yl-acetamide;
2-[4-(difluoromethyl)-6-[2-[4-[[4-(hydroxymethyl)-1-piperidyl]methyl]phenyl]ethynyl]-7-methyl-indazol-2-yl]-2-(6,7-dihydro-5H-pyrrolo[1,2-c]imidazol-1-yl)-N-thiazol-2-yl-acetamide; and
2-(4-(difluoromethyl)-6-((4-((4-(hydroxymethyl)piperidin-1-yl)methyl)phenyl)ethynyl)-7-methyl-2H-indazol-2-yl)-2-(6′,7′-dihydrospiro[cyclopropane-1,5′-pyrrolo[1,2-c]imidazol]-1′-yl)-N-(thiazol-2-yl)acetamide;
or a pharmaceutically acceptable salt thereof.

30. The method according to claim 21, wherein the allosteric EGFR inhibitor is 2-[4,7-dichloro-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide or a pharmaceutically acceptable salt thereof.

31. The method according to claim 21, wherein the allosteric EGFR inhibitor is 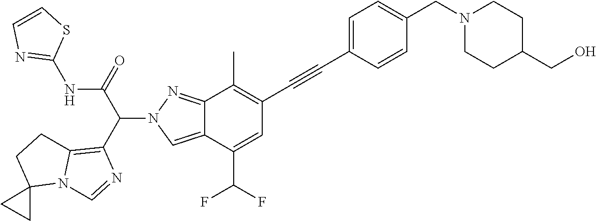 [SMILES: Cc1c(C#Cc2ccc(CN3CCC(CO)CC3)cc2)cc(C(F)F)c2cn(C(C(=O)Nc3nccs3)c3ncn4c3CCC43CC3)nc12]
or a pharmaceutically acceptable salt thereof.

32. The method according to claim 21, wherein the cancer is non-small cell lung cancer.

33. The combination according to claim 1, wherein the compound of formula (I) is 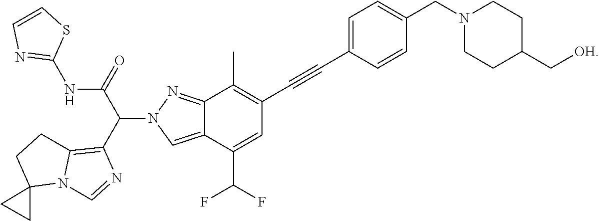 [SMILES: Cc1c(C#Cc2ccc(CN3CCC(CO)CC3)cc2)cc(C(F)F)c2cn(C(C(=O)Nc3nccs3)c3ncn4c3CCC43CC3)nc12]
